(19) United States
(12) Patent Application Publication
(10) Pub. No.: US 2007/0207191 A1
(43) Pub. Date: Sep. 6, 2007

(54) PHARMACEUTICAL COMPOSITIONS AND METHODS TO ACHIEVE AND MAINTAIN A TARGETED AND STABLE COPPER STATUS AND PREVENT AND TREAT COPPER-RELATED CENTRAL NERVOUS SYSTEM DISEASES

(76) Inventors: Steve H. KANZER, Ann Arbor, MI (US); George J. Brewer, Ann Arbor, MI (US); Nicholas Stergis, Ann Arbor, MI (US); John S. Althaus, Ann Arbor, MI (US); Charles L. Bisgaier, Ann Arbor, MI (US)

Correspondence Address: HAYES SOLOWAY P.C., 3450 E. SUNRISE DRIVE, SUITE 140, TUCSON, AZ, 85718, US

(21) Appl. No.: 11/621,962

(22) Filed: May 7, 2007

Related U.S. Application Data
(60) Provisional application No. 60/757,672, filed on Jan. 10, 2006.
(60) Provisional application No. 60/765,812, filed on Feb. 7, 2006.

(51) Int. Cl. A61K 33/32; A61K 31/315; A61K 9/70

(52) U.S. Cl. 424/449; 424/643; 514/494

(57) ABSTRACT

Compositions and methods are provided that comprise improved means to achieve and maintain a targeted level of copper status in persons in order to treat and prevent copper associated diseases.

### CROSS REFERENCE TO RELATED APPLICATIONS

This application claims priority from U.S. Provisional Application Ser. No. 60/757,672, filed Jan. 10, 2006 and U.S. Provisional Application Ser. No. 60/765,812, filed Feb. 7, 2006.

### FIELD OF THE INVENTION

The present invention relates to pharmaceutical products and methods for treating excessive metal buildup or metal malabsorption in animals and humans. The invention has particular applicability to treatment of Wilson's Disease in humans and will be described in connection with such utility, although other utilities are contemplated including treatment of other neurological diseases caused by excessive copper accumulation in the brain and other organs and/or intraday fluctuations in levels of free copper in the serum and central nervous system (CNS), including but not limited to Alzheimer's Disease, Parkinson's Disease, amyotrophic lateral sclerosis (ALS) or Lou Gehrig's Disease, dementia, Huntington's Disease, and schizophrenia, as well as neuromuscular diseases associated with abnormal accumulation of copper associated proteins in the body, such as juvenile and sporadic inclusion body myositis and myositis of the elderly, and cardiovascular diseases such as atherosclerosis, stroke, and peripheral vascular disease. The invention also may be used for the treatment of neurological and psychiatric manifestations of hepatic diseases associated with impaired liver copper excretion, such as colangitis, hepatitis and cirrhosis, for example, in which free or loosely bond serum or CSF copper is elevated.

### BACKGROUND OF THE INVENTION

Copper is a trace element that is essential to life. Despite its essentiality, however, copper also is an extremely reactive oxidative species that has the potential to be very toxic to cells, proteins, and organ systems such as the liver, brain and vasculature. In order to deliver and utilize copper in the body on demand wherever it is needed, mammalian systems have developed an elaborate regulatory network of highly specific, homeostatic, copper-binding cuproproteins that serve to properly scavenge, store, transport, chaperone and excrete copper while minimizing the potential for copper to inadvertently oxidize or reduce proteins and lipids. Many different cuproproteins have been identified and their functions have been elucidated over the years. Examples of such cuproproteins include, but are not limited to, matrix metalloprotein, ceruloplasmin, copper/zinc superoxide dismutase, amyloid precursor protein, apolipoprotein E, tau, homocysteine, albumin and chaperone for copper zinc superoxide dismutase, to name a few.

One of the most problematic and potentially toxic sources of copper for humans is the abundance of toxic copper ions that exists in drinking water systems. Unlike the copper found in food, which is bound to proteins and the absorption of which is relatively easily regulated by the intestines and the slow dissolution from food during digestion, the copper in drinking water occurs in the form of cupric ion (Cu⁺²) in either an unbound form or in a form complexed only loosely with organic ligands. Copper ions are generally more bioavailable in water than they are in food; there may be components in food that can influence the metabolism, absorption and mobilization of copper in human diets.

Absorption of Copper by the Human Body

In humans, dietary copper is absorbed from the stomach and small intestine. In one study, about 65 percent of an oral dose of Cu⁶⁴ as copper acetate was absorbed from the gastrointestinal tract of humans (range 15-97 percent) (Weber et al., 1969; Strickland et al., 1972). Absorption efficiency appeared to be inversely correlated with copper level in the diet (Turnland et al., 1989, 1998). Orally administered Cu⁶⁴ rapidly appears in the plasma (Beam and Kunkel, 1955).

Dorner et al. (1989) found that full-term, breast-fed human infants, with a copper intake of 114 μg/kg-day, retained 88 μg/kg-day of copper, representing an absorption value of ˜77 percent. Copper retention decreased with age. At two weeks of age, 130 μg/kg-day was retained, and at age 16 weeks, 64 μg/kg-day was retained. In comparison, mean relative retention in infants fed copper-fortified formula was 52 percent. Copper absorption in infant rhesus monkeys using Cu⁶⁷ ranged from 50-70 percent, similar to the values found for full-term human infants (Lonnerdal et al., 1996). Studies in rats show that copper absorption is very high during the neonatal period, but that it decreases by the weaning period (Lonnerdal et al., 1985). Using perfused rat intestines, Varada et al. (1993) found that copper absorption was linear and nonsaturable in infant and weanling rats, and copper absorption was saturable in adolescent rats. Suckling rats had considerably higher tissue copper concentrations than weanling or adolescent rats. Citrate, a dietary food-ligand found in human and cow milk, has been shown to have a positive effect on copper absorption in animal models (Shah, 1981).

Olivares et al. (2002) administered an oral supplementation of 80 μg Cu (as copper sulfate solution)/kg daily for 15 days to a group of Chilean infants aged 1-3 months (n=20); one half of the group (n=19) received no supplementation. At the end of the trial, copper absorption was measured by using orally administered Cu⁶⁵ as a tracer and fecal monitoring of recovered Cu⁶³. No major difference in the percentage of copper absorbed was observed between the two groups. Mean (+SD) copper absorption at one month of age was 83.6+5.8 percent and 74.8+15.2 percent for the unsupplemented and supplemented infants, respectively. The authors concluded that the experimental design of the study was inadequate because copper intakes were too low to “trigger homeostatic adaptation of intestinal absorption.”

Copper absorption in the gastrointestinal tract has been studied in rats and hamsters. Absorption takes place from the stomach and duodenum in rats (Van Campen and Mitchell, 1965) and from the lower small intestine in hamsters (Crampton et al., 1965). Copper absorbed from the gastrointestinal tract may be bound to amino acids or may be in the form of ionic copper. Copper becomes bound to metallothionein in the intestine and may be either absorbed or sloughed off back into the intestinal lumen.

The existence of a protein source (plant or animal protein), amino acids, carbohydrates and/or ascorbic acid can affect copper availability (Gibson, 1994; Lonnerdal, 1996). Competition with zinc and cadmium affects copper absorption from both diet and drinking water (Davies and Campbell, 1977; Hall et al., 1979). Ascorbic acid may alter the metallothionein binding site. High dietary ascorbic acid has been shown to interfere with absorption of copper in guinea pigs (Smith and Bidlack, 1980), but this does not appear to be a factor at the usual ascorbic acid doses in humans (Jacob et al., 1987). Phytates and fiber interfere with copper absorption by forming complexes with copper (Gibson, 1994). The amount of copper stored in humans, which is mainly in the liver, does not appear to affect copper absorption (Strickland et al., 1972). There do not appear to be any available studies of copper absorption in humans by inhalation.

Batsura (1969) observed copper oxide in alveolar capillaries after rats were exposed to welding dust from a pure copper wire. No studies of the rate or extent of absorption of copper through intact human skin were found, but as copper can cause contact dermatitis, some absorption must occur (ATSDR, 1990). Pirot et al. (1996) studied the absorption of copper and zinc through human skin in vitro. Skin absorption is not likely to contribute significantly to total copper absorption.

Distribution of Copper in the Human Body

Copper is transported in the plasma and is bound to ceruloplasmin, albumin or amino acids (Cousins, 1985). Ceruloplasmin is a cysteine-rich glycoprotein with many free sulfhydryl groups that serve as binding points for metals. Ceruloplasmin can bind copper or zinc, but has a stronger affinity for copper (Cousins, 1985). Ceruloplasmin is synthesized on membrane-bound polyribosomes of liver parenchymal cells and secreted into the plasma. Copper that enters the portal circulation from the intestine is transported directly to the liver. Copper released from the liver is transported in the bloodstream to other organs, including the kidney and brain. The synthesis of ceruloplasmin is controlled by interleukin-I via glucagon or glucocorticoid (Cousins, 1985). Circulating copper levels are elevated in pregnant women because hormonal changes associated with pregnancy stimulate ceruloplasmin synthesis (Solomons, 1985). Ceruloplasmin levels may be useful as an indicator of copper status (Mendez et al., 2004).

Recently, several copper transporters involved in copper uptake and transport by cells have been identified (Bauerly et al., 2005). Copper transporter-1 (Crt1) is a copper import protein that is copper-specific, and is believed to mediate copper uptake into the small intestine (Lee et al., 2002). Crt1 is expressed in the enterocytes of the small intestine and in enterocyte-like Caco-2 cells in culture (Klomp et al., 2002; Kuo et al., 2001). The copper efflux protein, ATP7A, is thought to mediate copper efflux across the plasma membrane during copper excess in transfected cells (Petris et al., 1996). Menkes disease, characterized by excessive copper accumulation in the intestine and systemic copper deficiency, is a consequence of a defect in ATP7A (Schaefer and Gitlin, 1999). ATP7B, with functional similarity to ATP7A, exports copper into bile for excretion (Roelofsen et al., 2000). ATP7B is localized primarily in the liver with lower expression found in the intestine, kidney and placenta (Lockhart et al., 2000). A defect in ATP7B results in Wilson's disease, characterized by copper toxicity (due to liver copper accumulation as a result of impaired biliary copper excretion) and liver damage.

Metabolism and Excretion of Copper

The liver and intestine play key roles in copper metabolism. Copper is taken up by hepatocytes from the portal circulation. Inside the hepatocytes, copper is bound to metallothionein, a protein that also binds zinc, iron and mercury. Copper can be released from hepatocytes into the general circulation to be transported to other tissues, or it can be excreted from the liver in bile (Cousins, 1985). The major route of excretion is in the bile. Only a small amount is excreted in the urine (Cousins, 1985). Biliary excretion in human infants is immature at birth, and the lack of an effective excretion mechanism may place infants at increased risk for copper toxicity.

Physiological/Nutritional Role of Copper

Because copper is an essential nutrient, an understanding of its numerous physiological roles in the body is essential for understanding the deleterious effects of copper deficiency or excess. Copper is essential for hemoglobin synthesis and erythropoiesis (Solomons, 1985; Harris, 1997). Copper deficiency can therefore lead to anemia. Copper deficiency can likewise lead to abnormalities of myelin formation, with attendant effects on the nervous system (Solomons, 1985; Harris, 1997). Nervous system effects, including dementia, have been observed in individuals with copper deficiency or excess (Solomons, 1985; Harris, 1997). Effects on catecholamine metabolism likewise are involved in the nervous system abnormalities. Other physiological functions that involve copper include leukopoiesis, skeletal mineralization, connective tissue synthesis, melanin synthesis, oxidative phosphorylation, thermal regulation, antioxidant protection, cholesterol metabolism, immune and cardiac function, and regulation of glucose metabolism. Since all of these physiological processes involve copper, any of them can be affected by the availability of copper in the body or in specific tissues. In general, deleterious effects may occur in any of these physiological processes due to either deficiency or excess of copper in the systems affected (Solomons, 1985; Harris, 1997).

The specific copper requirements of infants have not been well established. In infants, copper is an essential mineral that is required for normal growth, and the development of bone, brain, immune system, and red blood cells (Hurley et al., 1980). Full-term infants are believed to possess adequate copper stores at birth to last through weaning, but premature infants, prone to copper deficiency, must be given higher provisions of copper to compensate for inadequate copper stores (Lonnerdal, 1998).

Recommended Daily Allowances (RDAs) of copper were not provided in earlier RDA compilations because of difficulty in determining the values (NAS, 1989). Homeostatic mechanisms result in variable absorption and excretion of copper as dietary intake is manipulated, complicating mass balance calculations in dietary studies. However, in the most recent publication of recommended allowances (FNB, 2000), copper nutritional requirements have at last been established. Table 1 (below) shows the Dietary Reference Intake (DRI) values for copper for various age groups, broken down into Estimated Average Requirements (EAR), Recommended Dietary Allowances (RDA), and Tolerable Upper Intake Levels (UL) (FNB, 2000). Values for infants were provided only as Adequate Intake values, based primarily on the content of copper in human milk. The AI values are 200 μg/day for infants 0-6 months of age, and 220 μg/day for infants at 7-12 months; an estimated UL for infants could not be established (FNB, 2000).

TABLE 1
Recommended Daily Copper Dietary
Reference Intakes by Sex/Age

| | | Estimated | Recommended | Tolerable |
|---|---|---|---|---|
| | | Average | Dietary | Upper Intake |
| Age | | Requirement | Allowance | Level |
| (years) | Sex | μg/day | μg/day | μg/day |
| 1-3 | F/M | 260 | 340 | 1,000 |
| 4-8 | F/M | 340 | 440 | 3,000 |
| 9-13s | F/M | 540 | 700 | 5,000 |
| 14-18 | F/M | 685 | 890 | 8,000 |
| 18+ | F/M | 700 | 900 | 10,000 |
| Pregnant, 14-18 | F | 785 | 1,000 | 8,000 |
| 19+ | | 800 | 1,000 | 10,000 |
| Lactating, 14-18 | F | 985 | 1,300 | 8,000 |
| 19+ | | 1,000 | 1,300 | 10,000 |

Values from FNB, 2000.

Copper intake values from food and supplements, developed from the NHANES III nationwide survey (1988-1994), are shown in Table 2 (below). The NHANES III table and Continuing Survey of Food Intakes of Individuals (C SFII) indicate that intake of copper is adequate for the great majority of the population in all age and sex groups. However, results for some younger sex/age groups indicate as much as 10 percent of the population consuming less than the RDA of copper. On the other hand, considering the tendency for underreporting of food intakes, particularly for teenagers (Champagne et al., 1998), the lower end of the distribution curve is likely to be inaccurate.

TABLE 2
Copper Intake (mg/day) from Food and Supplements
Versus the Recommended Dietary Allowance (RDA)^{a}

| | | | Percentile | | | | | | | | RDA |
|---|---|---|---|---|---|---|---|---|---|---|---|
| Age and Sex | | | 5 | 10 | 25 | 50 | 75 | 90 | 95 | 99 | (mg/day) |
| 2-6 | mo | M/F | 0.3 | 0.4 | 0.5 | 0.7 | 0.9 | 1.1 | 1.2 | 1.6 | 0.20 |
| 7-11 | mo | M/F | 0.3 | 0.4 | 0.5 | 0.7 | 0.9 | 1.2 | 1.3 | 1.7 | 0.22 |
| 1-3 | yr | M/F | 0.3 | 0.4 | 0.5 | 0.7 | 1.0 | 1.3 | 1.7 | 2.9 | 0.34 |
| 4-8 | yr | M/F | 0.59 | 0.67 | 0.80 | 0.95 | 1.14 | 1.36 | 1.61 | 3.06 | 0.44 |
| 9-13 | yr | F | 0.64 | 0.72 | 0.86 | 1.04 | 1.26 | 1.54 | 1.84 | 3.23 | 0.70 |
| | | M | 0.88 | 0.94 | 1.05 | 1.21 | 1.41 | 1.61 | 1.78 | 3.13 | 0.70 |
| 14-18 | yr | F | 0.64 | 0.75 | 0.89 | 1.08 | 1.32 | 1.64 | 1.96 | 3.32 | 0.89 |
| | | M | 0.79 | 0.89 | 1.11 | 1.42 | 1.80 | 2.28 | 2.71 | 3.56 | 0.89 |
| 19-30 | yr | F | 0.77 | 0.83 | 0.95 | 1.12 | 1.38 | 1.82 | 3.03 | 3.84 | 0.90 |
| | | M | 1.37 | 1.43 | 1.56 | 1.69 | 1.86 | 2.12 | 3.55 | 4.44 | 0.90 |
| 31-50 | yr | F | 0.72 | 0.81 | 0.95 | 1.17 | 1.52 | 2.32 | 3.09 | 4.19 | 0.90 |
| | | M | 0.89 | 1.03 | 1.29 | 1.61 | 2.09 | 2.93 | 3.67 | 4.87 | 0.90 |
| 51-70 | yr | F | 0.61 | 0.68 | 0.84 | 1.07 | 1.48 | 2.92 | 3.25 | 4.22 | 0.90 |
| | | M | 0.75 | 0.87 | 1.09 | 1.43 | 1.98 | 3.00 | 3.65 | 5.02 | 0.90 |
| 71+ | yr | F | 0.58 | 0.65 | 0.80 | 1.02 | 1.37 | 2.94 | 3.21 | 3.79 | 0.90 |
| | | M | 0.72 | 0.83 | 0.99 | 1.26 | 1.66 | 2.89 | 3.41 | 4.61 | 0.90 |

| | | | | | | | | | | |
|---|---|---|---|---|---|---|---|---|---|---|
| Pregnant | F | 0.71 | 0.82 | 1.07 | 1.62 | 3.11 | 4.03 | 4.39 | 5.56 | 1.0 |

^{a}Breast-feeding infants and children, and eight individuals reporting greater than 150 mg/day of copper from supplements excluded from the analysis. RDA values from FNB, 2000.

While the majority of persons may be able to cope with chronic exposure to toxic copper ions contained in drinking water without showing signs of disease, there are certain rare diseases in which a person's copper transport and metabolic pathways are affected by genetic mutations, such as Wilson's disease and Menkes disease. The genetic mutations responsible for Wilson's disease and Menkes disease were identified for the first time in the 1990's by several groups. In addition to the rare Wilson's disease patients, there have been some published reports of elevated levels of serum copper in the elderly (Madaric et. al., Physiol Res, 1994; 43(2): 107-11 and Ghayour-Mobarhan et. al., Ann Clin Biochem, 2005 September; 42(Pt 5):364-75), which may be attributable to a compromised ability to properly process and excrete copper via the liver, into the bile, and ultimately through the stool. Accordingly, this patient population may share a sensitivity to copper which is similar to that of Wilson's disease patients. Elevated levels of non-ceruloplasim bound copper have been reported in elderly Alzheimer's disease patients (Squitti, et. al.).

Wilson's disease is characterized by a mutation of the gene encoding the P-type ATPase, called ATP7B. Due to the impairment of ATP7B, Wilsons' disease patients are unable to adequately process, transport and excrete copper through the normal bile ducts of the liver. In the case of normal subjects, copper that is newly introduced is expected to first bind to available engogenous cuproproteins having the highest affinity for copper, such as metallothionein, superoxide dismutase and albumin. Free copper ions are relatively rare in serum, but copper which is “loosely bound” to various proteins and peptides can be substantial and elevated in Wilson's disease patients and potentially also in other metabolically compromised groups such as Alzheimer's disease patients, mild cognitive impairment (MCI) patients, schizophrenia patients, dementia patients, and the elderly.

In the 1990's, the genetic defect responsible for Menkes Disease was identified as a mutation to another P-type ATPase, ATP7A. Menkes Disease is characterized by abnormally low levels of available copper, due to the failure of intestinal cells to release copper, and results in various developmental abnormalities.

The following are estimated contributions to total serum copper content of various serum proteins in normal patients: ceruloplasmin (650-750 ug/L, 65-70%), albumin (120-180 ug/L, 12-18%), transcuprein (macroglobulin) (90 ug/L, 9%), ferroxidase II (10 ug/L, 1%), extracellular SOD and histidine rich glycoproteins (<10 ug/L, <1%), blood clotting factors V and VIII (<5 ug/L, <0.5%), extracellular metallothionein and anime oxidase (<1 ug/L, <0.1%), 15-60 kDa components (40 ug/L, 4%), small peptides and amino acids (35 ug/L, 4%), and, ultimately, unbound or “free” copper ions (0.0001 ug/L, approx. 0%) (Linder, M C Biochemistry of Copper (ed.) 1991; Linder, M C (2001), Copper and Genomic Stability in Mammals., Mutat. Res. 475, 151-52).

As noted, a substantial proportion of circulating serum copper is bound to 15-60 kDa proteins (approx. 4%) and small peptides and amino acids (an additional approximated 4%). Such small proteins and peptides are capable of transporting loosely bound copper across the blood brain barrier, thereby creating an environment wherein copper may exist in excess and may therefore be detrimental to the health of neurons. In order to maintain a healthy copper homeostasis and to protect from extracellular lipid peroxidation and intracellular oxidation, neurons may upregulate a variety of copper binding proteins, including APP, Amyloid beta, tau, BACE1 and apoE, all of which are upregulated in Alzheimer's disease (and intracellularly in a similar fashion in the neuromuscular disease, inclusion body myositis).

Solubilized copper or copper loosely bound to small ligands, such as that commonly found in tap water, is highly bioavailable (up to 65%) and, due to water fluxes in the intestines, has the capacity to overwhelm the copper homeostasis mechanisms of the gastrointestinal enterocytes and liver, and enter the portal and systemic circulation in a potentially toxic form loosely bound to albumin and other low kinetic copper binding proteins. It is an object of the present invention to provide compositions, formulations, agents and methods to protect the individual from such toxic fluxes, as further described herein.

Wilson's Disease

In the case of untreated Wilson's disease patients, body copper continues to accumulate, ultimately overwhelming the high affinity cuproproteins. Residual copper either remains free and unbound or loosely bound to cuproproteins having low affinities to copper. This pool of free, unbound or loosely bound copper is free to circulate and may cross the blood brain barrier, damaging nerve cells due to its reactivity and pro-oxidant capacity. Clinically, the various cuproproteins serve as a reservoir for copper and will generally be released based upon an inverse correlation with each cuproprotein's individual affinity for copper. The so called “loosely bound” cuproproteins include albumin and homocysteine, for example. Such cuproproteins serve as potential toxic pools of available copper as compared to high affinity copper binding proteins, such as ceruloplasmin and cu/zn superoxide dismutase.

When the ability to excrete copper is impaired because of genetic disease or because there is hepatic impairment due to fibrosis or bile flow, the higher affinity cuproproteins, such as metallothionein, will ultimately become fully saturated and the copper binding process will continue with the lesser affinity copper binding proteins, such as homocysteine and albumin, until each of these protein pools become saturated as well. In the case of Wilson's patients, total copper overwhelms the capacity of high affinity cuproproteins to adequately bind and sequester it. The excess elevated body copper, so-called “free” or “unbound” or “loosely bound” copper, builds up in the body and is available to cross the blood brain barrier into the central nervous system. Over time, this copper toxicity damages or destroys organ systems such as the brain and liver.

Treatment for Wilson's Disease

Where a network of cuproproteins is abnormal as a result of genetic abnormality or aging, for example, then copper in the body may not be properly bound and sequestered by cuproproteins and therefore may not be properly maintained. Toxicities and oxidative capacities are most pronounced when copper is present in the body in its so-called “free”, “unbound” or “loosely bound” forms. Such free copper can be toxic to various organ systems, such as the liver and the brain, for example. The classic example of such a disease is Wilson's disease. Treatment varies for patients that are initially presenting, and they are generally treated on an acute or induction basis, with potent copper chelators and complexors, such as tetrathiomolybdate, penicillamine or trientine, each of which is intended to either remove available free copper from the body or render it unavailable for further damage. Following initial acute or induction treatment, patients will be switched to a chronic or maintenance therapy on a long term basis, generally for the remainder of their lives. Agents commonly used for chronic or maintenance therapy include those that maintain a state of copper malabsorption, such as zinc acetate (Brewer). Zinc acetate is available as a prescription and is marketed in the United States under the tradename Galzin® and in Europe under the tradename Wilzin®. Other zinc salts available without a prescription in the United States have been reportedly used by Wilson's patients for long term maintenance therapy with varying degrees of success. Examples of such other salts include, but are not limited to, zinc carbonate, zinc sulfate, zinc gluconate, zinc oxide, zinc chloride and zinc stearate, for example.

Zinc was used for the comprehensive treatment of Wilson's disease including initial treatment (Hoogenraad et al., Lancet, 2:1262-1263 [1978]; Hoogenraad et al., Eur. Neurol., 18:205-211 [1979]; and Hoogenraad et al., J. Neurol. Sci., 77:137-146 [1987]). However, zinc was not ideal for initial therapy (by itself) because it is rather slow acting. Thus, it takes approximately two weeks to achieve intestinal metallothionein induction and a negative copper balance in Wilson's patients (Yuzbasiyan-Gurkan et al., J. Lab. Clin. Med., 120:380-386 [1992]). At the two week point, zinc immediately reverses the +0.54 mg daily (positive) copper balance that these patients average, but the negative copper balance induced is rather modest, averaging −0.35 mg daily (negative) copper balance (G J Brewer et al., J. Trace Elem. Exp. Med., 3:227-234 [1990]; G J Brewer et al., Amer. J. Med. Sci., 305:(4)199-202 [1993]). Due to this low rate of copper removal, it takes as long as six months of zinc therapy to bring urine copper and nonceruloplasmin plasma copper (the potentially toxic copper measured in the blood), down to subtoxic levels. Tetrathiomolybdate (TM) is a more effective blocker of copper absorption than zinc, since zinc acts only in those areas of the small intestine where metallotionein can be induced. In contrast, TM works all up and down the gastrointestinal track. The other advantage of TM over zinc is that TM acts immediately. It does not have a lag period required for the induction of metallothionein.

Ultimately, such chronic maintenance therapies fail in some patients due to chronic and acute stomach and esophageal irritation and nausea commonly associated with such agents, difficulty in predicting effects and in setting an appropriate dosing regimen, and the need to continuously monitor free serum copper levels in order to assure that they are maintained within the normal range. The variability of effect of such agents depends upon the timing of administration as it relates to the timing of meals, difficulties in maintaining adequate patient compliance given the daily multiple dosing regimen, possible stomach irritation and the need to time each dose at least one hour prior and three hours after meals, as well as the need to assure compliance for the entire remaining lifetime of the patient.

Other Disorders Associated with Elevated Levels of Free Copper and/or Accumulation of Copper.

Other disorders are associated with elevated levels of loosely bound, free copper, and/or accummulation of copper includes headaches, hypoglycemia, increased heart rate, nausea, anemia, hair loss, nephritis, autism, depression, hallucinations, hyperactivity, insomnia, disperception of the senses, paranoia, personality changes, psychosis, schizophrenia, mild cognitive impairment, detachment from reality, atherosclerosis, stroke, tauapathies and synucleinopathies, nonalcoholic steatohepatitis, multiple sclerosis, Alzheimer's, Parkinson's, dementia, ALS and autism which are given as exemplary. In addition, diseases associated with increased inflammation and fibrosis are associated with normal to elevated levels of free copper and can be alleviated by a reduction of these levels via copper reduction intervention.

Alzheimer's Disease

As human life span has significantly expanded over the last century, Alzheimer's disease and other neurodegenerative diseases will have a growing impact on the quality of life for a large proportion of the population. For example, Alzheimer's disease is a leading cause of dementia in the elderly, affecting 5-10% of the population over the age of 65 years. See A Guide to Understanding Alzheimer's disease and Related Disorders, edited by Jorm, New York University Press, New York (1987). Alzheimer's disease often presents with a subtle onset of memory loss followed by a slow progressive dementia over several years. The prevalence of Alzheimer's disease and other dementias doubles every five years beyond the age of 65. See 1997 Progress Report on Alzheimer's disease, National Institute on Aging/National Institute of Health. Alzheimer's disease now affects 12 million people around the world, and it is projected to increase to 22 million by 2025 and to 45 million by 2050. See Alzheimer's Association Press Release, Jul. 18, 2000.

The complexity of the brain's architecture and chemistry, and the complexity of these neurodegenerative brain diseases, especially Alzheimer's disease, has hampered the development of a model that mimics many of the changes seen in the human brain. Such a model is needed in order to identify drugs or other agents that might be useful in treating, preventing or reversing the effects of such diseases.

Alzheimer's disease is histopathologically characterized by the loss of particular groups of neurons and the appearance of two principal lesions within the brain, termed senile plaques and neurofibrillary tangles. See Brion et al., J. Neurochem. 60:1372-1382 (1993). Senile plaques occur in the extracellular space. A major component of senile plaques is beta-amyloid (A-beta), a naturally secreted but insoluble peptide formed by cleavage of amyloid precursor protein (APP). A-beta is a fragment close to the carboxyterminal domain of APP.

Neurofibrillary tangles are intraneuronal accumulations of filamentous material in the form of loops, coils or tangled masses. They are most abundantly present in parts of the brain associated with memory functions, such as the hippocampus and adjacent parts of the temporal lobe. See Robbins Pathologic Basis of Disease, Cotran et al., 6.sup.th ed. (1999). Neurofibrillary tangles are commonly found in cortical neurons, especially in the entorhinal cortex, as well as in other locations such as pyramidal cells of the hippocampus, the amygdala, the basal forebrain, and the raphe nuclei.

Neurofibrillary tangles can also be found during normal aging of the brain, however, they are found in a significantly higher density in the brain of Alzheimer's disease patients, and in the brains of patients with other neurodegenerative diseases, such as progressive supranuclear palsy, postencephaltic Parkinson disease, Pick's disease, amylotrophic lateral sclerosis, etc. Robbins Pathologic Basis of Disease, Cotran et al., 6th ed. (1999), p. 1330. Previous studies suggest that, among other things, neurofibrillary tangles may significantly contribute to the cognitive decline associated with the disease and also directly to neuronal cell death.

Ultrastructurally, neurofibrillary tangles are composed predominantly of paired helical filaments (“PHF”). A major component of PHF is an abnormally phosphorylated form of a protein called tau and its fragments. Robbins Pathologic Basis of Disease, Cotran et al., 6th ed., W. B. Saunders Company (1999), p. 1300.

The tau protein (also referred to as “native tau”) is a microtubule-associated phosphoprotein that stabilizes the cytoskeleton and contributes to determining neuronal shape. See Kosik & Caceres, Cell Sci. Suppl. 14:69-74 (1991). Tau has an apparent molecular weight of about 55 kDa. The protease cathepsin D cleaves tau protein at neutral (cytoplasmic) pH resulting in tau fragments—one of which has a molecular weight of approximately 29 kDa (referred to by some authors as “tau fragment”). See, e.g., Bednarski & Lynch, J. Neurochem. 67:1846-1855 (1996); Bednarski & Lynch, NeuroReport 9:2089-2094 (1998). Both the tau protein and 29 kDa tau fragment can be phosphorylated. In a normal brain, the tau protein and tau fragment typically exist in an unphosphorylated, or dephosphorylated state. However, in neurofibrillary tangles, both tau protein and tau fragment can be found in an abnormally phosphorylated state, a hyperphosphorylated state. The 29 kDa tau fragment is a major component of neurofibrillary tangles. Hyperphosphorylation impairs tau protein's ability to interact with microtubules.

Bednarski E, and Lynch G, J Neurochem 67:1846-55 (1996) cultured hippocampal slices with an inhibitor [N-CBZ-L-phenylalanyl-L-alanine-diazomethyl ketone (ZPAD)] of cathepsins B and L. The authors reported that this resulted in the degradation of high molecular weight isoforms of tau protein and the production of a 29-kDa tau fragment (tau 29).

Bednarski E, and Lynch G, Neuroreport 9:2089-2094 (1998) reported that incubating cultured hippocampal slices with chloroquine or with ZPAD resulted in increases in enzymatically active cathepsin D and the delayed appearance of a 29 kDa fragment of the tau protein. The authors proposed that inactivation of cathepsin L leads to induction of cathepsin D which leads to aberrant tau proteolysis and that such a pathway is likely to play an important role in brain aging.

In addition to the build-up of A-beta and of neurofibrillary tangles, increasing evidence has pointed to a link between lipid metabolism and Alzheimer's disease. Epidemiological studies found that patients with increased plasma low density lipoprotan cholesterol and cholesterol levels and cardiovascular diseases have an increased risk of Alzheimer's disease Kuo, Y -M, et al., Biochem. Biophys. Res. Comm. 252: 711-715 (1998); Jick, H., et al., Lancet 356:627-631 (2000). Also, long-term therapy with the 3-hydroxy-3-methylglutaryl coenzyme A reductase inhibitors appears to decrease the prevalence of Alzheimer's disease (Jick, H., et al., Lancet 356:627-631 (2000); Wolozin, B., et al., Arch. Neurol. 57:1439-1443 (2000)).

Consistent with a link to lipid metabolism, in vitro experiments have shown that cholesterol affects the generation and aggregation of beta amyloid (A-beta) (Bodovitz, S., and Klein, W. L., J. Biol. Chem. 271:4436-4440 (1996); Xu, H., et al., Proc. Natl. Acad. Sci. U S A 94:3748-3752 (1997); Howland, D. S., et al., J. Biol. Chem. 273:16576-16582 (1998)). Transgenic mice fed a high cholesterol diet also developed increased amounts of A-beta deposition (Refolo, L. M., et al., Neurobiol. Dis. 7:321-331 (2000)).

ApoB and apoE mediated transport of cholesterol into lysosomes is a critical step for cells to utilize these sterols, which is of particular importance for mature neurons that mainly rely on extracellular apoE mediated transport of cholesterol (Brown, M. S., and Goldstein, J. L., Annu. Rev. Biochem. 52:223-261 (1983)). Once in the lysosome, cholesterol and other lipids dissociate from ApoE before being utilized by the cell (Brown, M. S., and Goldstein, J. L., Annu. Rev. Biochem. 52:223-261 (1983)).

Changes in cholesterol levels may be involved in certain neurodegenerative diseases. For example, accumulation of insoluble A-beta1-42 has been found in Niemann-Pick type C (NPC) mutant cells (Yamazaki, T., et al., J. Biol. Chem. (2000)). These cells exhibit many pathologic characteristics, one of which is impaired intracellular transport of cholesterol (Millard, E. E., et al., J. Biol. Chem. 275:38445-38451 (2000)). Also, the ApoE4 isoform is a known risk factor for late-onset Alzheimer's disease.

Inhibition of cholesterol synthesis enhanced the phosphorylation of tau in dissociated cell cultures [ref. in (Sawamura, N., et al., J. Biol. Chem. 57:1439-1443 (2001))]. Likewise, hyperphosphorylation of tau has been demonstrated in cell cultures prepared from NPC mutant mice (Sawamura, N., et al., J. Biol. Chem. 57:1439-1443 (2001)). Gradually developing disturbances in lysosomes, which affect the sorting/trafficking of cholesterol from lysosomes and late endosomes, may, therefore, be contributors to the pathologies associated with neurodegenerative diseases and Alzheimer's disease.

U.S. Pat. No. 6,803,233 describes animal models of Alzheimer's disease in which cysteine protease inhibitors are capable of producing animal models of Alzheimer's disease including the hallmark neurofibrillary tangles (NFTs), composed of paired helical filaments of tau are concentrated. Such patent, however, does not describe the copper binding effects of cysteine nor homocysteine as it to the creation of available pools of low molecular weight copper-cysteine complexes capable of crossing the blood brain barrier and upregulating the production of APP, Aβ and tau proteins. In one aspect, the present invention involves formulations of zinc (and more preferentially, gastroretentive sustained release zinc) and folic acid to reduce and stabilize the systemic and CSF levels of low molecular weight copper cysteine complexes (such as copper-homocysteine) that the present inventors recognize as a contributing factor to neurodegenerative diseases such as Alzheimer's disease, Parkinson's disease and ALS, for example.

Prior Art Involvement of Copper in Alzheimer's Disease, Parkinson's Disease, ALS and other Disorders of the Central Nervous System (CNS).

At present in the prior art, there is a considerable amount of conflicting conclusions and hypotheses regarding the causative role of elemental copper and zinc in neurodegenerative diseases, such as Alzheimer's disease, Parkinson's disease, ALS and CJD. For example, it is known that many of the hallmark proteins associated with Alzheimer's disease are copper binding proteins, including amyloid precursor protein (APP), beta amyloid (Aβ) (including peptides 1-40 and 1-42), tau (including the paired helical fragments (PHFs) and neurofibrillary tangle (NFTs)), beta secretase (BACE1) and apolipoprotein E (apoE), including the three major human variants of apoE, apoE2, apoE3 and apoE4. Regarding the latter, it is noted by the inventors that apoE2, apoE3 and apoE4 differ only in regard to the presence or absence of cysteine residues at positions 112 and 158. it has previously been shown that apoE2, apoE3 and apoE4 differ in their ability to bind copper.

Epidemiological genetic studies indicate that presence of the apoE4 varient, having no cysteine residues at positions 112 and 158, increases the risk of AD, while apoE2, having two cysteine residues at positions 112 and 158, is considered to have protective benefit for Alzheimer's disease (as well as athlerosclerosis) as compared to the most common hum varient apoE3, which has only cysteine residue these positions.

Pursuant to published findings of the Framingham study, elevated levels of homocysteine have been implicated with an increased risk of Alzheimer's disease, although until the disclosure contained herein, the effects homocysteine as a low molecular weight copper binding protein capable of delivering, maintaining and slowing clearance of toxic, loosely bound, and therefore exchangable “free” copper in the CNS has not been previously described.

In addition, elevated levels of cholesterol have been implicated with an increased risk of Alzheimer's disease. In particular, elevated levels of oxidized cholesterol, 27S-hydroxy-cholesterol and/or 24S-hydroxy-cholesterol, have been found both in the CNS and circulation and circulation of Alzheimer's patients (as well athlerosclerosis). In addition to Alzheimer's disease, elemental copper has also been hypothesized to play a role in other neurodegenerative diseases, such as, ALS, in which an mutant form of the copper/zinc binding protein, Cu/Zn superoxide dismutase (SOD1) has reduced ability to bind copper.

In Parkinson's disease, the copper, iron and aluminum binding protein, α-synuclein (AS) is known to be the major component of the neuronal and glial cytoplasmic inclusions known as Lewy Bodies widely considered as the hallmark lesions of both Parkinson's disease as well as the group of neurodegenerative disorders referred to as synucleinopathies.

Neural Tube Defects

Neural tube defects (NTDs) are major birth defects of the fetal brain or spine, and occurs when the neural tube (that later turns into the brain and spine) doesn't properly form, resulting in brain or spine damage. This occurs within the first few weeks a woman is pregnant, often before a woman knows that she is pregnant. Adequate intake of the B vitamin, folic acid by mothers prior to pregnancy has been shown to reduce the incidence of NTDs by up to 70% although the mechanism by which folic acid exerts this benefit has not yet been previously described. CDC, Folic Acid Now, CDC-NCEH99-0463, November 2005.

Spina bifida and anencephaly are two common types of NTDs. About 3,000 pregnancies in the United States are affected by spina bifida or anencephaly each year. Spina bifida occurs when the spine and back bones do not close all the way. When this happens, the spinal cord and back bones do not form as they should. A sac of fluid comes through an opening in the baby's back. Much of the time, part of the spinal cord is in this sac and it is damaged. Most children born with spina bifida live full lives, but they often have lifelong disabilities and need many surgeries.

Children born with spina bifida don't all have the same needs. Some children's problems are much more severe than others. Even so, with the right care, most of these children will grow up to lead full and productive lives.

Anencephaly occurs when the brain and skull bones do not form right. When this happens, part or all of the brain and skull bones might be missing. Babies with this defect die before birth (miscarriage) or shortly after birth.

Folic acid might help to prevent some other birth defects, such as cleft lip and palate and some heart defects. There might also be other health benefits of taking folic acid for both women and men. Low zinc and high levels of copper have been found in mothers of children with isolated cleft lip and palate. Hoyasz K K, Wiad. Lek., 58(7-8):382-5 (2005). Until the present invention, the role of folic acid in reducing pool of circulating serum copper bound to homocysteine has not been previously described.

Antioxidants

In another aspect the present invention provides formulations useful for lowering and maintaining steady systemic and CSF levels of free copper and iron which may be formulated and combined with one or more antioxidants, including, for example, vitamin C, vitamin E, Q10, omega 3 fatty acid, zinc-cysteine or combinations thereof.

Acetylcholine Esterase Inhibitors

Acetylcholinesterase inhibitors are highly regarded clinical agents for treating and improving senile dementia such as Alzheimer type senile dementia, or cerebrovascular dementia, attention deficit hyperactivity disorder and schizophrenia. In particular, donepezil hydrochloride (1-benzyl-4-[(5,6-dimethoxy-1-indanone)-2-yl]methylpiperidine hydrochloride) has been found to be useful as a acetylcholinesterase inhibitor in providing a desired pharmacological activity with minimum adverse side effects. In addition to donepezil hydrochloride, other known acetylcholinesterase inhibitors include rivastigmine (3-[1-(dimethylamino)ethyl]phenyl N-ethyl-N-methylcarbamate), metrifonate (dimethyl 2,2,2-trichloro-1-hydroxyethyl)phosphate), tacrine hydrochloride (1,2,3,4-tetrahydro-9-acridinamine), galanthamine hydrobromide, neostigmine, physostigmine etc. An object of the present invention as further described herein includes formulations that combine acetyl-cholinesterase inhibitors with agents selected from the group of zinc, zinc-cysteine tetrathiomolybdate, gastroretentive sustained release zinc formulations and sustained release formulations of other essential trace metals such as, copper and iron.

NMDA Receptor Antagonists

Excess activation of ionotropic glutamate receptors sensitive to N-methyl-D-aspartate (NMDA receptors) produces neuronal death and has been known to mediate various neurological diseases [Choi, Neuron 1:623-634 (1988)]. Glutamate, the excitatory neurotransmitter, is massively accumulated in brain subjected to hypoxic-ischemic injuries, which activates ionotropic glutamate receptors permeable to Ca²⁺ and Na⁺ and then causes neuronal death [Choi and Rothman, Annu Rev Neurosci 13:171-182 (1990)]. Antagonists of NMDA receptors remarkably attenuate brain injury following hypoglycemia, hypoxia, or hypoxic-ischemia [Simon, Swan, Griffiths, and Meldrum. Science 226:850-852 (1984); Park, Nehls, Graham, Teasdale, and McCulloch, Ann Neurol 24:543-551 (1988); Wieloch, Science 230:681-683 (1985); Kass, Chambers, and Cottrell, Exp. Neurol. 103:116-122 (1989); Weiss, Goldberg, and Choi, Brain Res. 380:186-190 (1986)]. Thus, NMDA receptor antagonists possess therapeutic potentials to protect brain against hypoglycemia, hypoxia, and hypoxic-ischemic injuries.

Excitotoxicity appears to contribute to neuronal degeneration following traumatic brain injury (TBI). Levels of quinolinic acid, an endogenous agonist of NMDA receptors, was increased 5- to 50-fold in human patients with TBI [E. H. Sinz, P. M. Kochanek, M. P. Heyes, S. R. Wisniewski, M. J. Bell, R. S. Clark, S. T. DeKosky, A. R. Blight, and D. W. Marion]. Quinolinic acid is increased in the cerebrospinal fluid and associated with mortality after TBI in humans [J. Cereb. Blood Flow Metab. 18:610-615, (1998)]. In animal models of brain trauma, levels of glutamate and aspartate were markedly increased [Faden, Demediuk, Panter, and Vink, Science 244:798-800 (1989)]. Glutamate release was also observed in rat spinal cord following impact trauma [Demediuk, Daly, and Faden. J Neurochem J. Neurochem. 52:1529-1536 (1989)]. NMDA receptor antagonists attenuate neuronal death following traumatic brain or spinal cord injuries [Faden, Lemke, Simon, and Noble. J. Neurotrauma. 5:33-45(1988); Okiyama, Smith, White, Richter, and McIntosh. J. Neurotrauma. 14:211-222 (1997)].

Glutamate plays a central role in the induction and the propagation of seizures [Dingledine, McBain, and McNamara, Trends. Pharmacol. Sci. 11:334-338 (1990); Holmes. Cleve. Clin. J. Med. 62:240-247 (1995)]. NMDA receptor antagonists were shown to act as anticonvulsants and antiepileptogenic drugs in various models of epilepsy [Anderson, Swartzwelder, and Wilson, J. Neurophysiol. 57:1-21 (1987); Wong, Coulter, Choi, and Prince. Neurosci. Lett. 85:261-266 (1988); McNamara, Russell, Rigsbee, and Bonhaus, Neuropharmacology 27:563-568 (1988)].

Amyotrophic lateral sclerosis (ALS) is accompanied by degeneration of both upper and lower motor neurons and marked by neurogenic atrophy, weakness, and fasciculation. While the pathogenesis of ALS remains to be resolved, excitotoxicity has been expected to participate in the process of ALS. In particular, ALS patients show increased levels of extracellular glutamate and defects in glutamate transport. Administration of excitotoxins mimicked pathological changes in the spinal cord of ALS patients [Rothstein. Clin. Neurosci. 3:348-359 (1995); Ikonomidou, Qin, Labruyere, and Olney J. Neuropathol. Exp. Neurol. 55:211-224 (1996)].

Antagonizing NMDA receptors appears to be applied to treat Parkinson's disease (PD). Several antagonists of NMDA receptors protect dopaminergic neurons from the neurotoxin MPTP (1-methyl-4-phenyl-1,2,3,6-tetrahydropyridine) [Lange, Loschmann, Sofic, Burg, Horowski, Kalveram, Wachtel, and Riederer. Naunyn Schmiedebergs Arch. Pharmacol. 348:586-592 (1993); Brouillet and Beal. Neuroreport. 4:387-390 (1993)]. NMDA receptor antagonists also ameliorate levodopa-induced dyskinesia and thus can improve the therapeutic effects of levodopa [Papa and Chase, Ann. Neurol. 39:574-578 (1996); Marin, Papa, Engber, Bonastre, Tolosa, and Chase, Brain Res. 736:202-205 (1996)]. Two NMDA receptor antagonists, memantine and dextromethophan, have been proved beneficial in treating PD patients [Verhagen, Del Dotto, Natte, van den Munckhof, and Chase, Neurology 51:203-206 (1998); Merello, Nouzeilles, Cammarota, and Leiguarda. Clin. Neuropharmacol. 22:273-276 (1999)].

Huntington's disease (HD) is a progressive neurodegenerative disease predominantly affecting small- and medium-sized inteneurons but sparing NADPH-diaphorase neurons containing somatostatin and neuropeptide in the striata. These pathological features of HD are observed in the striatal tissues following the intrastriatal injections of quinolinic acid or cultured striatal neurons exposed to NMDA, raising the possibility that NMDA receptor-mediated neurotoxicity contributes to selective neuronal death in HD [Koh, Peters, and Choi, Science 234:73-76 (1986); Beal, Kowall, Ellison, Mazurek, Swartz, and Martin, Nature 321:168-171 (1986); Beal, Ferrante, Swartz, and Kowall. J. Neurosci. 11:1649-1659 (1991)].

Thus, another object of the present invention as further described herein includes formulations that combine NMDA antagonists, such as, memantine and flupirtine with agents selected from the group of zinc, zinc-cysteine, tetrathiomolybdate, gastroretentive sustained release zinc formulations and sustained release formulations of other essential trace metals such as, copper and iron.

Free Radicals and Brain Diseases

Free radicals are produced in degenerating brain areas following hypoxic-ischemia or traumatic brain and spinal cord injuries [Hall and Braughler, Free Radic. Biol. Med. 6:303-313 (1989); Anderson and Hall, Ann. Emerg. Med. 22:987-992 (1993); Siesjo and Siesjo, Eur. J. Anaesthesiol. 13:247-268(1996); Love, Brain Pathol. 9:119-131 (1999)]. Antioxidants or maneuvers scavenging free radicals attenuate brain damages by hypoxic-ischemia or traumatic injuries [Faden, Pharmacol. Toxicol. 78:12-17 (1996); Zeidman, Ling, Ducker, and Ellenbogen, J. Spinal. Disord. 9:367-380 (1996); Chan, Stroke 27:1124-1129 (1996); Hall, Neurosurg. Clin. N. Am. 8:195-206 (1997)]. Extensive evidence supports that free radicals can be produced in brain areas undergoing degeneration in neurodegenerative diseases possibly due to point mutations in Cu/Zn superoxide dismutase in ALS, decreased glutathione and increased iron in PD, accumulation of iron in AD, or mitochondrial dysfunction in HD [Rosen, Siddique, Patterson, Figlewicz, Sapp, Hentati, Donaldson, Goto, O'Regan, and Deng. Nature 362:59-62 (1993); Jenner and Olanow, Neurology 47: S161-S170 (1996); Smith, Harris, Sayre, and Perry, Proc. Natl. Acad. Sci. U.S.A. 94:9866-9868 (1997); Browne, Ferrante, and Beal, Brain Pathol. 9:147-163 (1999)]. Accordingly, antioxidants have been neuroprotective against such neurodegenerative diseases [Jenner, Pathol. Biol. (Paris.) 44:57-64 (1996); Beal, Ann. Neurol. 38:357-366 (1995); Prasad, Cole, and Kumar. J. Am. Coll. Nutr. 18:413-423 (1999); Eisen and Weber, Drugs Aging 14:173-196 (1999); Grundman, Am. J. Clin. Nutr. 71:630S.-636S (2000)].

Zinc and Brain Diseases

Zn²⁺ mediates neurodegenerative process observed in seizure, ischemia, trauma, and Alzheimers disease (AD). The pharmacological activation of kainate receptors by administration of kainate, a seizure-inducing excitotoxin, causes the translocation of Zn²⁺ into postsynaptic degenerating neurons in several forebrain areas [Frederickson, Hernandez, and McGinty. Brain Res. 480:317-321 (1989)].

The prior art has focused on the reduction of zinc in the central nervous system by means of chelation and/or blockade. Blockade of Zn²⁺ translocation with Ca-EDTA attenuates neuronal loss following a transient forebrain ischemia or traumatic brain injury [Koh, Suh, Gwag, He, Hsu and Choi, Science 272: 1013-1016 (1996); Suh, Chen, Motamedi, Bell, Listiak, Pons, Danscher, and Frederickson, Brain Res. 852:268-273 (2000)]. Zn²⁺ is observed in the extracellular plaque and degenerating neurons in AD, which likely contributes to neuronal degeneration in AD [Bush, Pettingell, Multhaup, Paradis, Vonsattel, Gusella, Beyreuther, Masters, and Tanzi, Science 265:1464-1467 (1994); Suh, Jensen, Jensen, Silva, Kesslak, Danscher, and Frederickson. Brain Res. 852:274-278 852 (2000)]. Bush A I, et. al. and the company Prana are clinically testing the zinc/copper chelator, cliquinol and PBT-01 for the treatment of Alzheimer's disease and Huntington's disease. Yet another aspect of the present invention, and on which is in apparent contradiction to such approach, includes the use of gastroretentive sustained release formulations of zinc to increase intestinal, systemic and cerebral spinal fluid (CSF) levels of zinc and induce intestinal metallothienein and thereby reduce and/or maintain stable levels of loosely bound, “free” copper in the systemic circulation and CSF.

Cholesterol

Sterols are structural lipids present in the membranes of all eukaryotic cells. These lipids are rigid and characterized by a four ring hydrocarbon steroid nucleus. Sterols are required not only to impart membrane fluidity, but also serve as the precursors for a variety of products with specific biological activities. For example, cholesterol, an amphipathic sterol with a polar hydroxyl head group and nonpolar hydrocarbon body (the steroid nucleus), is the major sterol found in animal tissues. Cholesterol is an essential molecule, playing a critical role in the structural integrity of cell membranes, a precursor for steroid hormones and serves as a precursor for bile acids. Cholesterol is synthesized in all organs but especially the liver from acetate and further obtained via dietary intake.

Although cholesterol is a requisite molecule, high levels of blood cholesterol carried in the apoB containing lipoprotein or hypercholesterolemia has been implicated in atherosclerosis, heart attack, and stroke (Schultheis, 1990; Mitchell, 1990). Hypercholesterolemia, if not controlled, is one of several conditions that can lead to coronary artery disease. Coronary artery disease is the leading cause of death in the United States, accounting for approximately 600,000 deaths per year. Thus, the need exists for methods of treatment that can reduce cholesterol levels and methods to screen patients at risk for high cholesterol.

Possible targets for treatment are transcription factors involved in cholesterol metabolism. One such set of factors, nuclear receptors, are ligand-activated transcription factors that govern aspects of every major developmental and metabolic pathway (reviewed in Kastner et al., 1995; Mangelsdorf et al., 1995). For example, the LXRs were first identified as “orphan” members of the nuclear receptor superfamily whose ligands and functions are unknown (Willy and Mangelsdorf, 1998). The LXRs have recently been shown to be activated by a specific class of naturally occurring, oxidized derivatives of cholesterol, including 22(R)-hydroxycholesterol, 24(S)-hydroxycholesterol, and 24,25(S)-epoxycholesterol (Janowski et al., 1996; Lehmann et al., 1997). Oxysterols are concentrated in tissues where cholesterol metabolism and LXR expression are high, such as liver, brain, and placenta (Lavy et al., 1977; Spencer et al., 1985; Lutjohann et al., 1996).

LXRs function as heterodimers with the retinoid X receptors (RXRs), and thus, the RXR/LXR complex can be activated by both RXR ligands (i.e., rexinoids) and oxysterols (Teboul et al., 1995; Willy et al., 1995; Janowski et al., 1996). Two LXR proteins (alpha and beta.) are known to exist in mammals. The expression of LXRalpha is restricted, with highest levels in the liver (hence, the name liver X receptor) and lower but significant levels in kidney, intestine, spleen, and adrenals (Apfel et al., 1994; Willy et al., 1995). LXR.beta. expression is more widespread and has been found in nearly every tissue examined (Shinar et al., 1994; Song et al., 1994).

The pattern of expression of LXRs and their oxysterol ligands first suggested that these receptors may have a role in cholesterol metabolism. Cholesterol has three essential metabolic fates in mammals: esterification (for transport or storage), and conversion into steroid hormones, or bile acids. Since steroid hormone synthesis is known to be governed by the orphan nuclear receptor steroidogenic factor-1 (SF-1) (Parker and Schimmer, 1997), it is possible that LXRs are involved in bile acid synthesis (Janowski et al., 1996). A likely target for any bile acid inducer is cholesterol 7.alpha.-hydroxylase (Cyp7a), the rate-limiting enzyme in the classical bile acid synthesis pathway (Janowski et al., 1996; Lehmann et at., 1997). The Cyp7a promoter contains a functional LXR response element that can be activated by RXR/LXR heterodimers in an oxysterol- and retinoid-dependent manner (Lehmann et (al., 1997). The formation of bile acids is one of two major pathways for the catabolism and excretion of cholesterol in mammals (Russell and Setchell, 1992). Perturbations in this pathway may lead to a variety of disorders, including cholesterol gallstones, atherosclerosis, and Alzheimer's disease. Together, these observations have raised an interesting possibility that LXRs may function as transcriptional control points in bile acid metabolism.

The RXR protein of RXR homo- and heterodimers has been observed to be regulated by 9-cis retinoic acid, which binds to the carboxy-teminus of RXR (Mangelsdorf and Evans, 1995). RXR can form heterodimers with numerous other proteins in the nuclear receptor superfamily, including LXR. Depending on the receptor protein that dimerizes with RXR, and the ligands present, the resulting effects of the heterodimer on transcription can vary. Synthetic retinoids have been found to selectively bind and activate RXRs (U.S. Pat. No. 5,780,676 and U.S. Pat. No. 5,455,265). U.S. Pat. No. 6,835,866 describes the use of RXR-specific ligands, such as LG100268 to improve hepatic clearance of cholesterol.

The potential to modulate lipid concentrations in vivo, by targeting proteins of the nuclear hormone receptor superfamily with specific ligands may be useful in the treatment of various diseases related to lipid metabolism. For example, high blood cholesterol levels are associated with coronary disease. Lowering dietary cholesterol intake can significantly reduce cholesterol levels in most people. However, lowering dietary intake of cholesterol often is not enough, as certain individuals sustain high cholesterol blood levels due to inefficient endogenous cholesterol homeostasis. Thus, the ability to reduce blood cholesterol levels would prove to be extremely beneficial to these individuals. Currently, there are various drugs that are administered to treat hypercholesterolemia and other abnormal blood lipid levels. For example, cholestyramine and colestipol are resins that bind bile acids in the intestinal tract, causing the liver to increase its production of bile acids and thus lower the cholesterol levels, by converting cholesterol into bile acid. Nicotinic acid, gemfibrozil, probucol, as well as statins such as atorvastatin, lovastatin, pravastatin are commonly used to lower blood lipid levels.

In addition, bile acids, such as, ursodeoxycholic acid (UDCA) 17.beta.-(1-methyl-3-carboxypropyl)etiocholane-3.alpha., 7.beta.diol. are known to improve biliary function. No major toxicity is known to be associated with UDCA (W. H. Bachrach et al., Dig. Dis. Sci., 30, 642 (1985)). Thus, pharmaceutically acceptable salts and esters of UDCA include nontoxic esters of the free hydroxyl groups of UDCA with (C₁-C₄)alkanoic acids such as formic, acetic or propionic acid, phosphate esters of the OH groups, (C₁-C₄)alkyl esters of the free (C₂₄) carboxylic acid group and nontoxic alkali metal, ammonium or amine salts of the free carboxylic acid moiety. This ester and salts can be readily prepared from free UCDA by methods well known to the art. At least the diformate and diacetate esters are known compounds. See, The Merck Index (11th ed. 1989) at 1556. UDCA is commercially available in 300 mg hard gelatin capsules as ACTIGALL® from Summit Pharmaceuticals, Summit, N.J., and is prescribed for gallbladder stone dissolution.

The present invention in one aspect is based in part upon the unique finding that circulating levels of loosely bound or so-called “free” copper and iron in the systemic circulation and CSF are subject to substantial intraday peak fluctuations which are exacerbated by bolus administration of copper and iron in soluble form such as that contained in ordinary tap water as well as upon dissolution of immediate release copper or iron supplements. Furthermore, in individuals having reduced impaired hepatic or biliary function, such as is often the case in the elderly, such levels of free copper are maintained at elevated levels in the systemic circulation and CSF for longer periods of time due to impaired hepatic clearance, the combined effect of which makes them particularly susceptible to neurodegenerative disorders involving impaired copper and iron homeostasis in the CNS, such as, Alzheimer's disease, Parkinson's disease and ALS. Accordingly, a feature of the present invention as further described herein includes formulations that combine agents capable of improving hepatic and biliary clearance functions, such as, statins, including atorvastatin, RXR specific ligands, such as LG1002688, with agents selected from the group of zinc, zinc-cysteine, tetrathiomolybdate, gastroretentive sustained release zinc formulations and sustained release formulations of other essential trace metals such as, copper and iron so as to improve hepatic incorporation and clearance of free copper and iron in the systemic circulation and CSF of persons having potentially impaired hepatic and biliary function to treat or prevent neurodegenerative disorders, such as, Alzheimer's disease, mild cognitive impairment, Parkinson's disease, ALS and atherosclerosis caused by elevated levels and fluctuating levels of free copper and iron in the systemic circulation and CSF. Preferentially, such formulations may reduce the potential for hypocupremia and anemia through chroic zinc administration by the addition to such formulations of sustained release formulations containing copper, iron and/or other essential trace metals which preferentially will be bound to certain copper and iron binding excipients, such as whey, plant fibers, metallotheionein, dried milk or infant formula.

Parkinson's Disease

Parkinson's disease (PD) is a common neurodegenerative disorder and was first described by James Parkinson in 1817. The four primary diagnostic signs of the illness are resting tremor, bradykinesia, muscular rigidity and postural instability. These signs of motor deficiency result from the loss of dopaminergic neurons in the nigrostriatal system [Gibb, W., et al., J. Neurol. Neurosurg. and Psych., 51:745-52 (1988)].

PD is characterized by the formation of Lewy Bodies and death of dopaminergic neurons. [Adams D. et al., Principles of Neurology, 874-880, 3rd Edition, McGraw-Hill, N.Y., (1985)]. The neuropathological hallmark of PD is the LewyBody. Lewy Bodies are intracytoplasmic inclusions that occur in degenerating neurons which are composed of a dense core of filamentous and granular material surrounded by radical oriented filaments that have a diameter of 10-20 nm [Goedert, 20 M., et al., Curr. Op. Neurobio. 8:619-32 (1999)]. In general, the causes of PD are not known and there has been vigorous debate over the relative roles of genetics and environmental factors [Tanner, C., et al., JAMA, 281:341-6 (1999)]. Exposure to manganese precipitated a Parkinsonian syndrome in miners which also includes schizophrenia form behaviors. Some epidemiological studies have found an association between industrial exposure to iron and the incidence of PD [Corell J. M et al., Toxicol. Appl. Pharmacol., 80:467-72, (1985)], between incidence of PD and blood mercury levels [Ngim C. H. et al., Neuroepi., 8(3): 128-141 (1989)] and with death rates from PD and proximity to iron-related industrial processes [Rybicki A. et al., Mov Disord., 8(1):87-92_(1993)].

Alpha-Synuclein was originally identified as a protein that is upregulated and associated with neuron outgrowth during the critical period of Zebra finch song learning [George M., et al., Neuron, 15:361 (1995)]. Alpha-Synuclein is a ubiquitous protein that shares significant physical and functional homology to the protein chaperone, 14-3-3, and is particularly abundant in the brain (Ostrerova N. et al., J. Neurosci., 19:5782 (1990); Clayton D. et al., TINS 21:249 (1998)]. Alpha-Synuclein is normally-phosphorylated at serines 87 and 129. (Okochi M. et al., J. Biol. Chem., 275:390 (2000)]. Recent studies showed that mutations in alpha-synuclein can cause familial PD and that alpha-synuclein accumulates in LewyBodies. These discoveries suggest that alpha-synuclein participates in the pathophysiology of PD. (Spillantini M. et al., Nature, 388:839 (1997); Spillantini M. et al., PNAS USA, 95:6469 (1998); Jenner P. et al., Ann. Neurol., 44:S72 (1998)]. The only identified mutations associated with familial PD to date are the A53T and A30P variants in the alpha-synuclein protein (Goedert, M., et al., Curr. Op. Neurobio., 8:619-32 (1999); Papadimitriou, A., et al., Neurology, 52:651-4 (1999); Polymeropoulos, M., et al., Science, 276:1197-9 (1997)]. However, there has been much circumstantial evidence implicating oxidative stress in the etiology of the disease (Jenner, P., et al., Annual Neurol., 44:S72-84 (1998)].

A variety of experimental evidence suggests that Lewy Bodies interact with alpha-synuclein. For example, immunohistochemical studies indicate that Lewy Bodies stain strongly for alpha-synuclein and ubiquitin (Jenner, P., et al., Annual Neurol., 44:S72-84₋(1998); Markopoulou, K., et al., Annual. Neurol., 46:374-81 (1999); Spillantini, M., et al., Nature, 388:839-40 (1997); and Spillantini, M, et al., Proc. Natl. Acad. Sci. USA, 95:6469-73 (1998)]. In vitro experiments using recombinant protein suggest that the mutations, A53T and A30P, increase alpha-synuclein aggregation in comparison with the wild type alpha-synuclein (Conway, K., et al., Nature Med., 4:1318-20 (1998); Giasson, B., et al., J. Biol. Chem., 274:7619-22 (1999); Hashimoto, M., et al., Brain Res., 25 799:301-6 (1998)].

One of the important questions regarding alpha-synuclein aggregation and Lewy Body formation is whether these processes harm the cell. Lewy Bodies could either be inert tombstone markers that occur in response to free radical damage, or they might be toxic agents that harm the cell. Examples of both situations exist in the literature. Aggregated amyloid-beta (beta.) is toxic to neurons, while lipofuscin appears to be innocuous to cells (Behl, C., et al., Cell 77:817-27 (1994)]. The Huntington's protein presents an intermediate situation where the toxicity associated with Huntington's appears to precede aggregation, and aggregation of Huntington's protein might even be protective [Saudou, F., et al., Cell 95:55-66 (1998)]. Previous studies showed that transient over-expression of alpha-synuclein is toxic to a variety of cells, including two neuronal cell lines, SK-N-SH and PC12 [Ostrerova, N., et al., Neurosci., 19:5782-91 (1999)]. Consistent with this observation, Masliah and colleagues have recently shown that mice over-expressing alpha-synuclein show an age-related loss of dopaminergic terminals and motor impairment, which could be indicative of toxicity [Masliah, E. et al., Science, 287:1265-1269 (2000)]. These findings suggest that an increased rate of alpha-synuclein aggregation might contribute to the mechanisms of neurodegeneration in PD and other Lewy Body diseases.

Recent studies on transgenic animals also suggest that aggregation of alpha-synuclein is harmful to neurons. It was recently reported that dopaminergic dysfunction occurred in transgenic mice expressing wild type human alpha-synuclein [Masliah, E., et at. , Science, 287:1265-1269 (2000)]. Further, it was reported that Drosophila over-expressing alpha-synuclein exhibited dopaminergic dysfunction and dopaminergic neuronal death associated with development of alpha-synuclein aggregates [Feany, M B, et al., Nature 404:394-8 (2000)]. Evidence suggests that neurons with dopamine develop alpha-synuclein aggregates and degenerate as these aggregates development.

Recently, oxidative stress produced by iron and hydrogen peroxide has been shown to induce amyloid-like aggregate formation of alpha-synuclein in vitro [Hashimoto, M., et at., NeuroReport, 10:717-21 (1999); Paik, S., et al., Biochem. J., 340:821-8 (1999)]. Oxidative stress is thought to contribute to PD because dopamine, which is a strong free radical generator, is the principle neurotransmitter in the substantia nigra [Chiueh, C., et at., Adv. Neurol., 60:251-8 (1993); Jenner, P. et al., Ann. Neurol., 25 44:S72-84 (1998)]. In addition, iron, which also stimulates free radical production, accumulates in the substantia nigra with age [Jenner, P., et al., Ann. Neurol., 44:S72-84 (1998)]. Iron is deposited as hemosiderin granules in the cytoplasm, and mitochondria filled with ferritin granules have been observed in the neuronal and glial cells of the ventorlateral thalamus, caudate and lenticular nuclei and substantia nigra of Parkinsonian brains. [Earle M., J. Neuropathol. Exper. Neurol., 27(1):1-14, (1968); Asenjo A. et al., Rev. Neurologique, 121 (6):581-92, (1969); Riederer P., et al., J. Neurochem., 52(2):515-20, (1989)].

Nonalcoholic Steatohepatitis

Nonalcoholic steatohepatitis (NASH) involves the development of histologic changes in the liver that are comparable to those induced by excessive alcohol intake but in the absence of alcohol abuse. Macrovesicular and/or microvesicular steatosis, lobular and portal inflammation, and occasionally Mallory bodies with fibrosis and cirrhosis characterize NASH. NASH is also commonly associated with hyperlipidemia, obesity, and type II diabetes mellitus. Other clinical conditions characterized by hepatic steatosis and inflammation include excessive fasting, jejunoileal bypass, total parental nutrition, chronic hepatitis C, Wilson's disease, and adverse drug effects such as those from corticosteroids, calcium channel blockers, high dose synthetic estrogens, methotrexate and amiodarone. Thus, the term “nonalcoholic steatohepatitis” can be used to describe those patients who exhibit these biopsy findings, coupled with the absence of (a) significant alcohol consumption, (b) previous surgery for weight loss, (c) history of drug use associated with steatohepatitis, (d) evidence of genetic liver disease or (e) chronic hepatitis C infection. See, J. R. Ludwig et al., Mayo Clin. Proc., 55, 434 (1980) and E. E. Powell et al., Hepatol., 11, 74 (1990).

The pathogenesis of NASH is unknown. A correlation seems to exist between the degree of steatosis and the degree of fibrosis. For example, see I. R. Wanless et al., Hepatology, 12, 1106 (1990). Elevated hepatocellular free fatty acids may cause membrane injury with subsequent inflammation, possible cholestasis, and subcellular organelle dysfunction. Cell death and fibrosis follow persistent inflammation, and cirrhosis occurs if the injury continues. Steatohepatitis is now considered an important cause of end-stage liver disease and may be the cause of an unknown number of cases of clyptogenic cirrhosis. See E. E. Powell et al, cited above. Unfortunately, once cirrhosis is established, the only therapeutic modality available is orthotopic liver transplantation. Thus, effective therapy for nonalcoholic steatohepatitis is clearly needed.

Multiple Sclerosis

Elemental copper and is also known to play a role in the formation of myelin. Antigenic forms of myelin are also associated with the T-cell mediated lesions and myelin destructiion that is characteristic of multiple sclerosis.

Autism

Metabolic copper dysfunction has also been implicated in autism. Chuahan, A et. al, Life Sci. 2005 Oct. 8; 75(21):2539-49. Accordingly, yet another object of the present invention includes the treatment of autism with gastroretentive and/or sustained release formulations of zinc, copper and iron and other trace metals so as to maintain a targeted and steady level of free copper and iron in the systemic circulation and CSF of autistic children.

### SUMMARY OF THE INVENTION

The present invention in one aspect provides improved pharmaceutical compositions, kits and methods to improve the means to induce, monitor and safely maintain a state of copper malabsorption for extended periods in a person or animal in need of reducing, managing or maintaining low levels of free, unbound or loosely-bound copper, including, but not limited to, the indications of Wilson's disease, Alzheimer's disease, atherosclerosis, autoimmune diseases, oxidative stress, geriatric-related impaired copper excretion, liver disease, neurodegenerative disorders such as ALS, Parkinson's disease, and multiple sclerosis, as well as diseases associated with elevated levels of cuproproteins, such as schizophrenia. Also disclosed are specially formulated pharmacants, kits and dosing regimens intended to reduce the need to monitor patients for signs of hypercupronemia.

Thus, the present invention in one aspect provides pharmaceutical and over-the-counter products designed and formulated specifically to block absorption of copper and copper ions from the gastrointestinal tract, while also limiting the systemic bioavailability of such blocking agents.

### DESCRIPTION OF THE DRAWINGS

Further features and advantages of the present invention will be seen from the following detailed description taken in conjunction with the accompanying drawings wherein:

FIG. 1 is a plot of the dissolution profile of an ammonium tetrathromolybdate (ATTM) capsule in gastric juice in accordance with Example 13 hereof; and

FIG. 2 is a plot of ATTM formulations tested at room temperature and ambient humidity; Conditions were Mono (monohydrate lactose), Anydrous (anhydrous lactose, Quali (methyl cellulose capsules), Air (air atmosphere) and N2 (nitrogen atmosphere) in accordance with Example 14 hereof.

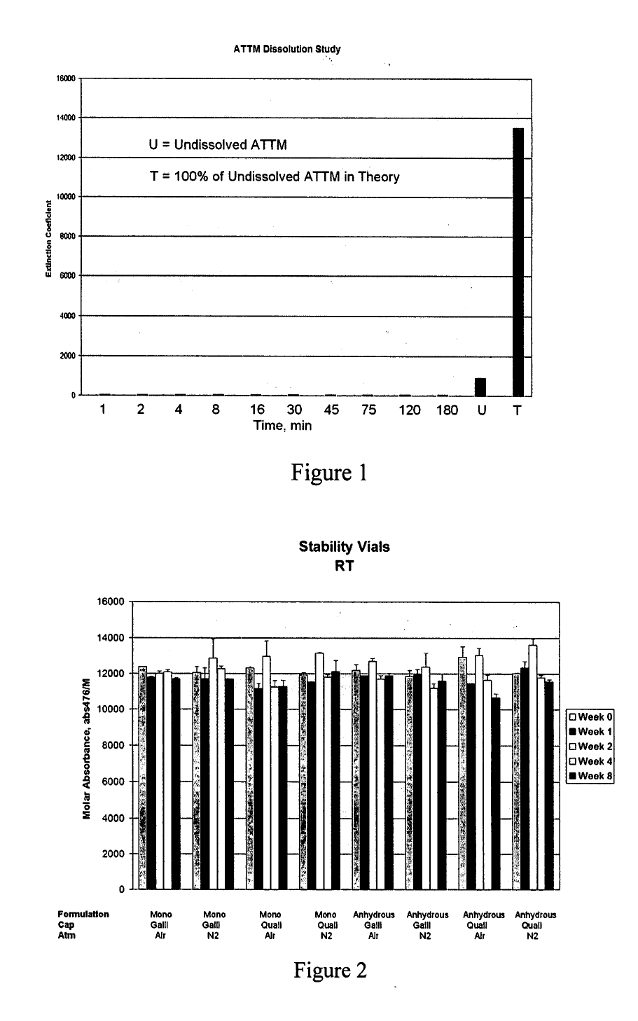

### DETAILED DESCRIPTION OF THE INVENTION

Novel Zinc Containing Formulations to Induce and Maintain a Targeted and Steady State Copper Balance

Zinc acetate formulated as an immediate release 25 mg and 50 mg capsule was developed and clinically tested as a maintenance therapy for the treatment of Wilson's disease. On Jan. 28, 1997 the FDA approved 25 mg and 50 mg forms of zinc acetate blended with corn starch and magnesium stearate formulated in immediate release gelatin capsules for maintenance treatment of patients with Wilson's disease who have been initially treated with a copper chelating agent. The recommended dosage for Wilson's disease patients is 50 mg taken three times a day (t.i.d.) at least one hour before meals and beverages (other than water) and at least one hour after meals and beverages (other than water).

Zinc blocks the intestinal absorption of copper from the diet and the re-absorption of endogenously secreted copper such as that from saliva, gastric juice and bile. Zinc induces the production of metallothionein in the enterocyte, a protein that binds copper and thereby prevents its serosal transfer into the blood. The bound copper is then lost in the stool following desquamation of the intestinal cells. The results of clinical studies in Wilson's disease patients with 50 mg immediate release zinc acetate capsules taken three times per day away from meals and beverages demonstrated that such a regimen induced a negative mean copper balance of −0.44 mg/day and an adequate mean Cu⁶⁴ uptake of 0.82% of the administered dose.

Importantly, once daily dosing of immediate release zinc acetate capsules does not provide adequate control of copper. As a result, t.i.d. and at a minimum, b.i.d. dosing is required for Wilson's patients. Combined with the necessity to administer zinc acetate capsules at least one hour before and at least one hour after meals, patient compliance is a major problem. Once daily dosing cannot be readily achieved simply by increasing the dose of zinc nor by utilizing a typical sustained release formulation since the induction of metallothienein in the intestinal lumen sufficient to block the absorption of copper is time dependent. It is estimated that exposure of the intestinal epithelium to zinc typically takes about two weeks of zinc administration at least two times a day to induce metallothienein sufficient to block absorption of copper. A sustained release formulation of zinc, while having some advantages as further described herein, will still only afford protection to the transient region of the intestines by which it is absorbed for a period of up six hours.

A further limitation of the currently FDA approved immediate release formulation of zinc acetate is the prevalence of gastric irritation associated with the release of a bolus zinc cation into the stomach.

Gastroretentive Agents that are Useful for the Maintenance of Copper Malabsorption

Pharmaceutical dosage forms which retain in the stomach for a prolonged period of time after oral administration, and release the active ingredient in a controlled manner, are important for delivery of a wide variety of drugs. Various pharmaceutical controlled release drug delivery systems with prolonged gastric retention time have been described in the literature. These involve different technologies.

The advantages of using drug delivery systems are many. Of major importance in controlled drug therapy is improved efficiency in treatment. Controlled drug therapy reduces the required frequency of administration, and single doses at periodic intervals are sufficient, resulting in improved patient compliance.

Current formulations of zinc acetate require dosing of at least two to three times per day in order to maintain a state of copper malabsorption. This is because the mechanism of action of zinc acetate is to induce the production of metallothionein in the gastrointestinal epithelium and thereby inhibit the subsequent absorption of copper from the areas in which metallothionein is expressed. It is estimated that once expressed in gastrointestinal epithelium, metallothionein will bind and thereby block the absorption of copper for a period of approximately 6 to 8 hours. Since the majority of copper, and especially water containing copper ions, is absorbed in the small intestines, it is necessary to refresh the exposure of the small intestinal epithelium several times per day in order to maintain the desired state of malabsorption.

Accordingly, it is an object of the present invention to provide for a formulation of zinc acetate that utilizes a gas troretentive mechanism so as to reduce the required frequency of dosing to once per day, while providing the small and large intestines with a continuous prolonged exposure of zinc acetate.

The present invention provides a benefit in diseases other than Wilson's disease, such as Alzheimer's disease, because it helps to achieve a comparable or greater level of copper malabsorption with an equal or lower amount of systemic zinc exposure. In addition to copper, zinc has been found to be bound to amyloid beta plaques in the brains of autopsy-confirmed Alzheimer's disease patients. Some studies suggest that reduction of zinc in the serum and CNS would therefore be a beneficial goal in the treatment of Alzheimer's disease (Bush A I). However, the long term effects of zinc in the serum and CNS have not been evaluated in Alzheimer's disease. Accordingly, the present invention, by providing a means to block copper absorption in the intestines while minimizing serum levels of zinc, reduces the uncertainty regarding the relationship between Alzheimer's disease and zinc. Zinc may play a beneficial role in the serum and CNS given its status as an anti-oxidant and zinc found in Alzheimer's plaques may not be causally related as in the case of copper.

To enable improved therapy in these cases, a gastroretentive pharmaceutical dosage form can be developed utilizing a number of alternative gastroretentive systems. After oral administration, such gastroretentive dosage form should retain in the stomach and release zinc acetate in a controlled and prolonged manner. Examples of gastroretentive dosage forms are floating dosage forms and dosage forms that expand, swell or unfold in the stomach.

The rationale for developing expandable drug delivery systems is based on the nature of the pyloric antrum that, by means of antiperistaltic motion, retropels large bodies away from the pylorus back to the fundus and body of the stomach, thus prolonging their gastric retention time (GRT). Such dosage forms should preferably be designed to undergo biodegradation or disintegration to enable their evacuation from the stomach.

U.S. Pat. No. 3,574,820 teaches the use of a gelatin matrix which hydrates in the stomach, gels, swells and cross-links with N-acetyl-homocysteine thiolactone to form a matrix too large to pass through the pylorus.

U.S. Pat. No. 4,207,890 discloses a drug dispensing device which comprises a collapsed, expandable imperforate envelope, made of a non-hydratable, body fluid and drug-permeable polymeric film, which contains the drug, and an expanding agent also contained within the polymeric envelope which, when in contact with body fluids, causes the envelope to expand to a volume such that the device is retained in the stomach.

U.S. Pat. No. 4,434,153 describes a device comprised of a matrix formed of a hydrogel that absorbs and imbibes fluid from the stomach, expands, and swells in order to retain in the stomach for an extended period of time, and a plurality of tiny pills dispersed throughout the matrix, having a drug-containing core and a fatty acid and wax wall surrounding the core.

A significant disadvantage of the devices of the prior art publications cited above is that they appear to ignore natural contractions of the stomach which may contribute to a rapid diminishing of size, leading to early removal of the device from the stomach. These devices lack the strength required to withstand the natural mechanical activity that includes contractions of the stomach.

U.S. Pat. Nos. 4,767,627, 4,735,804 and 4,758,436 present dosage forms of various geometries: continuous solid stick; tetrahedron; planar disc; multi-lobed flat device; and ring. The devices are compressible to a size suitable for swallowing, and are self-expandable to a size which prevents passage through the pylorus. They are sufficiently resistant to forces of the stomach to prevent rapid passage through the pylorus for a pre-determined period of time and erode in the presence of gastric juices. The devices are homogenous, namely they contain the same polymer constitution in different areas of the device. The tetrahedron presented in U.S. Pat. No. 4,735,804 is homogenous in its four lobes, which are attached to each other by a polymeric matrix.

Zinc acetate is incorporated into the device as a liquid solution or suspension, which may necessitate the addition of mentioned preservatives or buffering agents. Alternatively, the controlled release zinc acetate module may be tethered or glued to the device.

U.S. Pat. Nos. 5,002,772 and 5,443,843 disclose an oral drug delivery system having a delayed gastrointestinal transit, which releases the drugs contained therein in a controlled manner and which in their expanded form resist gastrointestinal transit. These delivery systems comprise one or more retention arms as a non-continuous compressible element, and an attached controlled release drug-containing device. The preferred configuration is a coil or a spiral. These systems must comprise at least two distinct parts (at least one retention arm and a controlled release device).

U.S. Pat. Nos. 5,047,464 and 5,217,712 describe a system comprising bioerodible, thermoset, covalently cross-linked, poly(ortho) ester polymers, which expand from a compressed state upon delivery thereof. The acidic environment of the stomach eventually results in the degradation of the polymers within the system, thus permitting its removal from the stomach. The system is characterized by high resiliency.

U.S. Pat. No. 5,651,985 describes a system devised from a mixture of polyvinyl-lactams and polyacrylates which are characterized by their high degree of swelling in the stomach resulting in its retention in the stomach for a prolonged period of time.

Finally, U.S. Pat. No. 6,685,962 describes a gastroretentive drug delivery system for the controlled release of an active agent in the gastrointestinal tract which comprises: (a) a single- or multi-layered matrix comprising a polymer that does not retain in the stomach more than a conventional dosage form selected from (1) degradable polymers that may be hydrophilic polymers not instantly soluble in gastric fluids, enteric polymers substantially insoluble at pH less than 5.5 and/or hydrophobic polymers and mixtures thereof; (2) non-degradable polymers; and any mixtures of (1) and (2); (b) a continuous or non-continuous membrane comprising at least one polymer having a substantial mechanical strength; and (c) a drug; wherein the matrix when affixed or attached to the membrane prevents evacuation from the stomach of the delivery system for a period of time.

A pharmaceutical composition can be formulated for oral, intravenous, intramuscular, subcutaneous, or inhalation administration as well as by other routes (i.e. enema, intranasal, intrathecal, etc). Advantages of orally administered pharmaceuticals (as a solution, suspension, tablet, capsule, etc.) include rapid therapeutic effect and patient convenience.

It is known in the art to orally administer a pharmaceutical in order to provide a direct effect on a target site within the gastrointestinal tract, as opposed to providing a therapeutic effect by absorption of the active ingredient of the pharmaceutical composition into the patient's circulatory system (i.e. antacids, laxatives). The controlled gastric retention of solid dosage forms of a pharmaceutical can be achieved by the mechanisms of mucoadhesion, flotation, sedimentation, expansion, or by the simultaneous administration of pharmacological agents which delay gastric emptying.

Mucoadhesion is the process whereby synthetic and natural macromolecules adhere to mucosal surfaces in the body. If these materials are then incorporated into pharmaceutical formulations, drug absorption by mucosal cells can be enhanced or the drug released at the site for an extended period of time. For synthetic polymers, such as the chitosans, carbopols and carbomers, the mechanism of bio/mucoadhesion is the result of a number of different physicochemical interactions. Biological bio/mucoadhesives, such as plant lectins, show specific interactions with cell surfaces and mucin and are seen as the “second generation” bioadhesives. (Woodley, J., Bioadhesion: new possibilities for drug administration?, Clin Pharmacokinet 2001;40(2):77-84). Thus, mucoadhesion acts to impart to orally administered dosage forms the ability to resist the strong propulsion forces of the stomach wall. The continuous production of mucous by the gastric mucosa to replace the mucous which is lost through the peristaltic contractions and the dilution of the stomach content can be overcome by use of mucoadhesion as a gastroretentive force. Mucoadhesive nanoparticulate systems, including liposomes and polymeric nanoparticles, have been evaluated. Mucoadhesive ability can be conferred on particulate systems by coating their surface with mucoadhesive polymers such as chitosan and Carbopol. The feasibility of such surface modification has been confirmed by measuring the zeta potential. Evaluation procedures include a particle counting method using a Coulter counter for polymer-coated liposomes. Mucoadhesive nanoparticles have been used for the oral administration of peptide drugs, and have been shown to be more effective with a more prolonged action as compared to non-coated systems (Takeuchi H., et al.). Mucoadhesive nanoparticulate systems for peptide drug delivery (Adv Drug Deliv Rev, Mar. 23, 2001; 47(1):39-54).

Mucoadhesive drug delivery devices offer several advantages over traditional dosage forms including the ability to optimize the therapeutic effects of a drug by controlling its release into the body. It has been shown that various types of poly(acrylic acid) (PAA) hydrogels are able to inhibit the hydrolytic activity of gastrointestinal enzymes, such as trypsin, resulting in an increase of the bioavailability of the drug. Acrylic-based polymers can be used for the attachment of mucoadhesive delivery systems to the mucosa. Polymer hydrogels modified by grafting mucophilic copolymers such as poly(ethylene glycol) (PEG) onto the back-bone chains of the polymer can promote the adhesive process. This is due to the ability of these grafted chains to diffuse from the network to the mucous layer. Films of P(AA-g-EG) can be synthesized by using UV-initiated free-radical solution polymerization. Different types of hydrogels can be synthesized with varying molar feed ratio of AA to PEG. The polymer hydrogels are characterized by mucoadhesion in order to quantify the effects of the PEG grafted chains on mucoadhesion. The bioadhesive bond strength can be determined using a tensile apparatus, and the work of adhesion thereby calculated. Hydrogels containing 40% AA and 60% PEG (40:60 AA/EG) can exhibit the highest mucoadhesion. These results can be attributed to the synergistic effects of both monomers. AA functional groups can permit the polymer to form multiple hydrogen bonds as well as to swell to a large degree. PEG tethers acted as mucoadhesive promoters. They penetrated into the mucosa and bridged the base hydrogel and the mucus. These results can also be interpreted in terms of the Huang-Peppas models (2002) of surface coverage and chain length effects in mucoadhesion.

Flotation as a retention mechanism requires the presence of liquid on which the dosage form can float, and it also presumes that the patient remains in an upright posture during the GRI, because in a supine position the pylorus is located above the stomach body and allows the accelerated emptying of floating material. Thus, flotation can be a basis principle for gastric retention of an oral formulation.

Sedimentation has been successfully used as a retention mechanism for pellets which are small enough to be retained in the rugae or folds of the stomach body near the pyloric region, which is the part of the organ with the lowest position in an upright posture. Dense pellets (of greater than approx. 2.4-2.8 g/cm³) trapped in rugae also tend to withstand the peristaltic movements of the stomach wall. Elemental zinc has a density of 7.165 g/cm³ which is 4.365 g/cm³ greater than the 2.8 g/cm³ threshold density necessary for gastroretention. Zinc's high specific density of 7.165 g/cm³ creates the opportunity to utilize up to approximately 60.9% of each pellet to incorporate other desirable, less dense materials to improve the function, safety, tolerability and effectiveness of the gastroretentive zinc pellets. Accordingly, the present invention describes an enteric-coated zinc pellet of sufficiently small size to become trapped in the rugae. Such an enteric coating, as described, provides the advantage of avoiding the potential for irritation to the stomach wall as a result of direct contact with the trapped zinc pellet. The enteric coating may be pH dependent and selected to prevent degradation of the coating and release of the zinc while the pellet is still in the low pH conditions of the stomach and/or pyloric region (pH of 1.2-3.5). The coating instead begins to degrade and release zinc in the substantially higher pH of the duodenum (pH of approx. 4.6-6.0), jejunum (pH of approx. 6.3-7.3) and/or colon or rectum (pH of approx. 7.9-8.0), or based upon the presence of bacterial flora ubiquitous to the colon and rectum (areas in which substantial quantities of water potentially containing high concentrations of copper are reabsorbed into the body). The pellets may be contained within a swallowable capsule that rapidly dissolves and releases the pellets upon entry into the stomach. The capsules may easily be varied according to the total amount of zinc contained, as well as by the mixture of number and type of pellets contained, to best suit the habits and dosing preferences of the patient and to provide greater confidence that the target “free” or “serum” copper will remain within the desired range.

Expansion has been shown to be a potentially reliable retention mechanism. Several devices have been described in the art which comprise features which extend, unfold or are inflated by carbon dioxide generated within the devices after administration.

These dosage forms are excluded from passage through the pyloric sphincter if they exceed a diameter of approximately 12-18 mm in their expanded state. Various mechanisms ensure the full reversibility of the expansion.

It is therefore an object of the invention to provide a controlled-release drug delivery system that retains a zinc acetate composition or device in the stomach for a sufficient period of time, while releasing zinc acetate therefrom.

Continuous Protection from Copper-Containing Drinking Water and Peak “Free Copper” Concentrations

Table 3 represents the copper content found in common dietary items and various organs.

TABLE 3
Copper Content of Dietary Items and Organs

| Item: | Copper Content (μg/mL or g wet weight) |
|---|---|
| Uncontaminated | 0.00001-0.001 |
| fresh water | (WHO limit = 2 ug/mL) |
| Vegetables | 0.3-3 |
| Fruits | 0.4-1.5 |
| Seeds and grains | 3-8 |
| Potatoes | 2.1 |
| Maize | 1.4 |
| Yeast | 8 |
| Shellfish | 12-37 |
| Freshwater fish | 0.3-3 |
| Saltwater fish | 2-3 |
| Liver and kidney | 4-157 |
| Heart | 4.5-4.8 |
| Muscle | 0.9-1.0 |
| Brain | 3.1-5.2 |
| Hair and Nails | 8-20 |

Linder, M C (Handbook of Copper Pharmacology and Toxicology (2003), p. 4.

As noted in Table 3, the proposed World Health Organization limit of copper in drinking water is 2,000 to 20,000 times the amount found in uncontaminated fresh water and 1,300 to 13,000 times the 1.3 mg/L limit established by the U.S. EPA pursuant to its Lead Copper Rule, revised (2000). Assuming an average drinking water consumption of 2 liters per day at the EPA limit, drinking water could provide 2.6 mg of copper per day. Assuming an average food intake of 1 kilogram per day, daily food consumption could provide approximately 1.0 mg of copper per day. However, the copper bound to proteins contained in foodstuffs, as well as the substantial amount of re-circulated copper entering the gastrointestinal system in its already processed and therefore protein-bound form (in the form of saliva, gastric juices, intestinal secretions, epithelial cell sloughing and bile) will likely be processed by the intestines in a more deliberate, enzyme-specific manner than the free, unbound, solubilized, potentially ionic copper contained in drinking water. A much greater percentage of the copper contained in drinking water will most likely be absorbed as a bolus because no digestion to free it from a food-legand complex is required. The intestinal cells will pass their bolus on the blood, where it is primarily loosely bound by albumin. Thus bolus effect may allow a significant part of the copper to bypass the liver, i.e., bypassing the intestines' evolutionary copper absorption and regulatory apparatus, and be picked up by the brain.

Copper in drinking water could enter the serum, bind to small peptides, and cross the blood barrier into the central nervous system, bypassing the normal blood brain barrier regulation of copper transport and homeostasis. Free or loosely bound copper in the CNS and the serum are believed to be at equilibrium. In addition to overwhelming the CNS with excess free copper, copper absorbed through drinking water is most likely absorbed in a bolus fashion as compared to copper absorbed through foods. The anticipated result is a much higher peak free copper concentration in the CNS as compared to the daily average. Such a peak free copper concentration in the CNS would presumably upregulate the copper binding proteins/ protective mechanisms such as APP, amyloid beta and tau. The copper molecules could also be in the ionic or cupric form and therefore of greater toxic potential.

It is an object of the present invention to provide continuous protection for copper-sensitive individuals, such as Wilson's disease, Alzheimer's disease, dementia and elderly patients, from the toxic effects of peak serum and CNS free copper concentrations by administration of special formulations of one or more continuous release copper malabsorption agents, such as zinc and ascorbic acid, and thereby reduce the effects associated with toxic free copper, especially the upregulation of protective cuproproteins in the CNS.

In a preferred embodiment, the gastroretentive pill or capsule utilizes a combination of different gastroretentive mechanisms to assure the broadest and least variable protection. For example, in addition to high density zinc salt pellets, capsules may be formulated with floating zinc salt-containing microparticles, mucoadhesive microparticles, mucoadhesive high density zinc-containing pellets, mucoadhesive high density enteric coated zinc-containing pellets, expanding gastroretentive systems containing zinc salts, as well as immediate release and/or non-gastroretentive zinc powder or enteric coated zinc microparticles. Such formulations will aid the prescribing doctor in estimating a recommended daily dosage to achieve a certain level of copper protection or copper malabsorption.

Enteric Pills, Capsules, Tablets or Microparticles Containing Zinc

Formulations of zinc acetate available as an FDA approved form, such as Galzin, are associated with poor compliance on the part of patients due to gastric irritability, which is believed to be associated with the ionic nature of zinc. Stomach irritability is associated with current formulations in an estimated 10% of patients.

Since copper-containing liquids, such as water, or the copper contained in foods, do not begin to absorbed in the human body until they reach the small intestines, it is not necessary to induce expression of metallothienein in the stomach in order to maintain a state of copper malabsorption. In addition to causing gastric irritability, immediate release formulations of zinc that are intended to induce a state of copper malabsorption also unnecessarily increase the systemic circulating levels of zinc and to other organ systems, such as the brain. Excess levels of zinc in the brain and central nervous system are implicated in certain neurodegenerative disorders in which excess copper is also implicated. Examples of such disorders include, but are not limited to, Alzheimer's disease, Parkinson's disease and amyotrophic later sclerosis (ALS).

Accordingly, it is yet an another object of the present invention to utilize an enteric coated formulation of zinc, so that the zinc-containing pill, capsule, tablet (or microparticles contained therein) does not begin to release the content of zinc until after it has fully transited the esophagus and stomach.

A typical enteric coating may be a polymeric material. Preferred enteric coating materials comprise bioerodible, gradually hydrolysable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating in the practice of the present invention to achieve delivery of the active drug to the lower gastrointestinal tract. The selection of the specific enteric coating material depends on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropyhnethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (e.g., those copolymers sold under the tradename “Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). Combinations of different coating materials may also be used to coat a single capsule. Particularly preferred enteric coating materials for use herein are those acrylic acid polymers and copolymers available under the tradename “Eudragit” from Rohm Pharma (Germany). The Eudragit series E, L, S, RL, RS and NE copolymers are available as solubilized in organic solvent, as an aqueous dispersion, or as a dry powder. The Eudragit series RL, NE, and RS copolymers are insoluble in the gastrointestinal tract but are permeable and are used primarily for extended release. The Eudragit series E copolymers dissolve in the stomach. The Eudragit series L, L-30D and S copolymers are insoluble in stomach and dissolve in the intestine, and are thus most preferred herein.

A particularly suitable methacrylic copolymer is Eudragit L, particularly L-30D and Eudragit 100-55. In Eudragit L-30D, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract. Another particularly suitable methacrylic acid polymer is Eudragit S, which differs from Eudragit L-30D in that the ratio of free carboxyl groups to ester groups is approximately 1:2. Eudragit S is insoluble at pH below 5.5, but unlike Eudragit L-30D, is poorly soluble in gastrointestinal fluids having a pH in the range of 5.5 to 7.0, such as in the small intestine. This copolymer is soluble at pH 7.0 and above, i.e., the pH generally found in the colon. Eudragit S can be used alone as a coating to provide drug delivery in the large intestine. Alternatively, Eudragit S, being poorly soluble in intestinal fluids below pH 7, can be used in combination with Eudragit L-30D, soluble in intestinal fluids above pH 5.5, in order to provide a delayed release composition which can be formulated to deliver the active agent to various segments of the intestinal tract. The more Eudragit L-30D used, the more proximal release and delivery begins, and the more Eudragit S used, the more distal release and delivery begins. It will be appreciated by those skilled in the art that both Eudragit L-30D and Eudragit S can be replaced with other pharmaceutically acceptable polymers having similar pH solubility characteristics.

The enteric coating provides for controlled release of the active agent, such that drug release can be accomplished at some generally predictable location in the lower intestinal tract below the point at which drug release would occur without the enteric coating. The enteric coating also prevents exposure of the hydrophilic therapeutic agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated capsules of the present invention allow optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The coating can, and usually does, contain a plasticizer to prevent the formation of pores and cracks that would permit the penetration of the gastric fluids. Suitable plasticizers include, but are not limited to, triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will usually contain approximately 10% to 25% by weight of a plasticizer, particularly dibutyl phthalate, polyethylene glycol, triethyl citrate and triacetin. The coating can also contain other coating excipients such as detackifiers, antifoaming agents, lubricants (e.g., magnesium stearate), and stabilizers (e.g., hydroxypropylcellulose, acids and bases) to solubilize or disperse the coating material, and to improve coating performance and the coated product.

The coating can be applied to the capsule using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms may be found in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6.sup.th Ed. (Media, Pa.: Williams & Wilkins, 1995). The coating thickness, as noted above, must be sufficient to ensure that the oral dosage form remains intact until the desired site of topical delivery in the lower intestinal tract is reached.

Copper Maintenance Therapies for Elderly and Geriatric Patients.

Several reports have found marked increases in serum copper in elderly patients, especially over the age of 75, (Madaric A, Ginter E, Kadrabova J, Physiol Res. 43;92: 107-11, 1994) as well as in elderly patients suffering from Alzheimer's disease (Squitti et. al., 2005). Since it has generally been concluded that mammalian copper homeostasis is mainly controlled at the level of excretion (Lindner M C, Biochemistry and molecular biology of copper in mammals, Handbook of Copper Pharmacology and Toxicology, edited by Massaro E J., Totawa N J: Humana, 2002, pp. 3-32.), an object of the present invention is directed towards achieving and maintaining a healthy level of copper status in elderly or geriatric patients through the restoration of normalized hepatic copper excretion. As opposed to approaches that seek to normalize copper by either blocking copper uptake or chelating or complexing copper, the present invention of normalizing hepatic excretion of copper has the important advantage of limiting the necessity to regularly monitor the copper status of each patient to assure that copper status remains within the targeted range, thereby avoiding copper toxicosis and hypercupronemia.

Agents that are useful for improving the excretion of copper in elderly patients include ursiodiol, anti-inflammatory agents and antifibrotics.

Depot Injection Formulations of Copper-Chelating, Copper-Complexing and Copper-Blocking Agents

An alternative to oral delivery of agents that reduce body copper is by means of a subcutaneous injection or subcutaneous implant. Liposomes have proven to be versatile carriers for the delivery of drugs. These carriers are biocompatible, since they are generally made from lipids commonly found in multilamelar systems and are biodegradable by the usual metabolic pathways. Multivesicular liposomes can be formulated with zinc to release in the body over the course of a month or more. As a result of their larger size (median diameter typically 10-30 μm), these multivesicular liposomes are not rapidly cleared by macrophages and can act as a drug depot, providing slow release of drugs for up to or more than one month. It is anticipated that such a depot formulation will improve compliance in Alzheimer's patients and nevertheless readily induce metallotheionein in the intestines sufficient to block absorption of copper from the intestines.

Alternatively, subcutaneous implants have been developed and utilized to deliver a drug of interest for up to a year or more. Such implants generally utilize polymer matrices that incorporate the drug of interest and permit its release in a time-dependent manner. Osmotic pumps have also been utilized in a similar fashion. An implant formulated with zinc could achieve easy compliance, especially in the case of Alzheimer's patients, and nevertheless readily induce metallothionein in the intestines sufficient to block absorption of copper from the intestines.

Alternatively, a transdermal patch could accomplish the same objectives as an implant and achieve steady state zinc in the blood stream.

It is anticipated that in the case of sustained release, depot formulation, implants and transdermal patches, incorporating a copper-blocking agent such as zinc will have the added benefit of reducing the peaks and throghs of loosely bound copper in the serum and other body compartments, thereby reducing the induction of copper transport proteins, such as, amyloid precursor protein, amyloid beta, and tau by neuronal cells at time points in which levels of intracellular copper are high. Accordingly, it is anticipated that, beyond the convenience of administration, such systems will have the added therapeutic benefit of reducing the total production of pathogenic copper transporters as compared to non-sustained release approaches, delivering a comparable average dose of zinc or other copper blocker, copper chelator or copper complexor.

Ascorbic Acid-Containing Formulations

Studies have shown that high levels of ascorbic acid (Vitamin C) can interfere with the absorption of copper. Accordingly, an object of the present invention is to utilize formulations including ascorbic acid to block the absorption of copper from the gastrointestinal tract.

Diseases Associated with Hyperhomocysteinemia

Elevated levels of serum homocysteine, a copper-binding protein having a molecular weight of 135 Daltons, have been associated with Alzheimer's disease, atherosclerosis and schizophrenia (the Framingham Study, NEJM 2003). Increased intake of folic acid, which reduces the circulating levels of homocysteine, has recently been reported to be beneficial for the prevention of the onset of Alzheimer's disease (the Baltimore Longitudinal Group, 2005), but only up to the daily intake limit, beyond which no additional benefit of folic acid is derived. It is possible that homocysteine represents an opportunistic pool of a circulating, low molecular weight, copper-binding protein that serves to maintain an elevated level of toxic, loosely bound copper that is also free to cross the blood brain barrier.

Accordingly, it is an object of the present invention to provide a method of treating schizophrenia and cardiovascular or other disease identified by elevated levels of homocysteine through the administration of a copper chelator, copper complexor or copper malabsorption agent, including zinc and gastroretentive formulations of zinc and zinc salts, and thereby reduce the circulating level of copper-bound homocysteine in the serum and CNS.

An object of the present invention is to utilize zinc-cysteine complexes, such as zinc-monocysteine described by Newsome in U.S. Pat. No. 6,586,611, as copper malabsorption agents for the treatment of Wilson's disease, schizophrenia, athlerosclerosis and neurodegenerative diseases associated with elevated levels of free copper, including Alzheimer's disease, ALS and Parkinson's disease.

Pharmaceutical Agents, Formulations and Methods to Protect Against Toxicities Associated with Fluctuations and Peak Levels of Toxic Free Copper and to Maintain a Stable Free Copper Status

While many individuals and research groups that together comprise the prior art agree that copper and other metals, such as iron, zinc, manganese and aluminum appear are likely to play a role in various neurodegenerative diseases, such as, Alzheimer's Parkinson's, ALS, such groups differ substantially with regard to the conclusions about whether copper and such other metals are in excess or deficient in such patients. As a result, such groups differ substantially in regard to their suggested intervention methods.

For example, Bayer, Phinney and Westaway have concluded that elemental copper is deficient in Alzheimer's patients and Bayer is believed to be in the process of initiating a clinical trials involving supplemental copper to treat Alzheimer's patients. Such approach is largely based upon the belief that the upregulation of intraneuronal copper binding proteins, such as, APP and Aβ are upregulated as a result of intraneuronal copper deficiency and attempts by the neuron to withhold the copper it has available. Bush, A I et. al are of the opinion that copper and zinc are both found in excess in the brains of Alzheimer's patients and they are recommending and testing the copper/zinc agent clioquinol on the basis that clioquinol is capable of crossing the blood brain barrier and binding copper and zinc in senile plaques and effectively either removing it, or “cementing it in”. Bush has recently assereted that clioquinol may be exerting its effects observed in animal models via maintaining the availability of copper in the CNS.

Other groups in the prior art have come to different conclusions, Sparks has found that copper in drinking water facilitates the formation of Alzheimer's plaques in rabbit models fed high cholesterol diets. More recently, Sparks has hypothesized that the clinical benefit of statins such as Lipitor® may be based upon their ability to lower copper as evidenced by a trend in lower ceruloplasmin levels in statin treated Alzherimer's patients. (Sparks, L, December 2005). Sparks suggests that avoidance of copper containing drinking water may be of benefit to Alzheimer's patients, although the mechanism by which copper in drinking water may be attributable to Alzheimer's disease has not been fully elucidated. Squitti et. al. has found both elevated levels of total copper (Squitti 2003) and elevated levels of copper not related to ceruloplasmin (Squitti 2005) in the serum of Alzhimer's patients, patients with mild cognitive impairment (MCI) compared to age matched controls. These results are in apparent contradiction to earlier published findings of Snaedal and other published studies measuring both total copper and ceruloplasmin levels in Alzheimer's patients compared to aged matched controls, where no such differences were observed.

Another object of the present invention is based upon the heretofore yet described observation that the levels of copper loosely bound to proteins other than ceruloplasmin (so-called free copper) in both serum and cerebral spinal fluid (CSF) are subject to substantial intraday fluctuation. Without not wishing to be bound by any particular theory, intraday fluctuation of free copper in the CNS could help explain the contradictory conclusions reached by different groups as to whether a goal of Alzheimer's disease treatment should be the reduction of copper intake or copper supplementation.

While free copper levels in the CNS and resulting intraneuronal free copper are elevated, it is likely that proteins capable of binding and precipitating intraneuronal free copper, such as, APP and the cleaved transmembrane peptide Aβ would be upregulated resulting in the precipitation of Aβ bound copper into the extraneuronal CSF. However, once free copper levels in the systemic circulation are reduced from peak levels (as a result of appropriate processing and excretion of copper by the liver into the bile and stool), the upregulation of APP is likely to a temporary intraneuronal deficiency of available copper necessary for proper neuronal metabolic functions that require copper. In such case, intraneuronal copper binding proteins such as tau may then be alternately upregulated in an effort to maintain an adequate supply intraneuronal available free copper.

Furthermore, such intraday fluctuations of potentially free copper in the serum and CNS are greatly exacerbated shortly following intake of water containing solubilized copper, copper ions and copper salts. Without being wished to be bound to any particular theory, the inventors attribute such free copper fluctuations to rapid influx copper containing water via the small intestines, the portal vein, serum plasma, crossing the blood brain barrier into the cerebral spinal fluid and brain. As opposed to dietary copper found in foods, the vast majority of which is gradually released becoming bioavailable as part of the normal digestive process in the intestines over the course of many hours, solubilized copper, such as that ordinarily found in tap water, exists in a free, unbound form and may rapidly enter the systemic circulation and CSF as a bolus shortly following influxes of water in the intestines due to the first pass effect. Such copper influxes have been shown to be increased in the presence of sodium (Wapnir) and thereby bypass or overwhelm the copper regulatory mechanisms of intestinal epithelia and liver by virtue of their inability to respond in a timely fashion via the upregulation of intestinal metallotheionein and hepatic ceruloplasmin. Water influxes of solubilized copper have been shown by Wapnir to be exacerbated in the presence of sodium thereby potentially implicating intestinal sodium channels as a potential entry point of solubilized copper in the portal vein and hepatic circulation. Copper bound to chylomicrons may also enter into the lymphatic circulation finally draining into the thoracic duct.

Again not wishing to be bound by any particular theory, in elderly persons, such as those susceptible to late onset Alzheimer's disease and Parkinson's disease, one would expect to find impaired hepatic function that is known to decrease with age. As a result, one would expect on average that the peak free copper concentrations in systemic circulation and CNS of elderly persons would not only be higher upon bolus administration of solubilized copper such as that found drinking water, but that duration of such peak free copper concentrations would be considerably extended, due to impaired hepatic clearance, and that the area under the curve (AUC) of free copper in the systemic circulation and CSF would be substantially greater than that of comparable younger persons following bolus administration of solubilized copper in drinking water.

### EXAMPLE 1

Four cohorts of patients of 12 or more patients each, one cohort with late onset Alzheimer's disease, one cohort with late onset Parkinson's disease, one cohort of normal elderly patients that are age matched to the Alzheimer's and Parkinson's cohort and one cohort of normal patients aged 20 to 40 are administered equal amounts of distilled or tap water containing the isotope Cu⁶⁴. Serial serum samples are obtained at time zero and every 20 minutes over the course of 2 to 24 hours. Serum samples are fractionated in three or more bead containing columns that have binding specificity for either ceruloplasmin, albumin and one or more other copper binding proteins, such as, homocysteine, and apoE. The beads are subsequently separately washed with distilled water and the eluted solution of each is measured and quantified for total radioactivity by means of standard protocols measuring total radioactivity indicating the total amount of Cu⁶⁴ present in such elution on an absolute and related on a percentage basis to both the total solution volume as well as quantity of ceruloplasmin, albumin or other proteins. The results show both a higher average level of peak Cu⁶⁴ levels and AUC of CU⁶⁴ in the albumin and other protein elutions in the Alzheimer's and Parkinson's patients compared to aged matched controls and young patients, as well as when age matched controls are compared to younger patients. The results demonstrate for the first time, that Alzheimer's patients, Parkinson's patients and elderly persons have a higher susceptibility to the toxic effects of peak levels of as result of solubilized copper contained in drinking water as well as a longer sustained period of exposure to free copper administered via drinking water. If serial CSF samples could be obtained the results would be similar and further demonstrate that because an equilibrium of free copper exists between the systemic circulation and CSF, as in the case of neurologically presenting Wilson's disease patients elevated levels of free copper in the systemic circulation are directly related to the clinical behavioral and neurological disturbances witnessed in such patients.

### EXAMPLE 2

The same experiment as described in Example 1 is repeated except all four cohorts of patients Alzheimer's patients, Parkinson's patients, age matched normals and young normals, an induction dose regimen of either (a) 100 mg/day of immediate release oral zinc acetate (Galzin®) is given for 14 days prior to and including day 0, (b) 100 mg/day of gastroretentive sustained release oral zinc acetate is given for 14 days prior to and including day 0, (c) 100 mg/day of gastroretentive sustained release oral zinc acetate in combination with 2 mg/day of oral sustained release copper and/or iron (either as a salt or bound to plant fiber, whey, metallotheionein, transferrin, dried milk, infant formula, or other natural copper or iron binding excipients) is given for 14 days prior to and including day 0, (d) 2 μg to 120 mg/day of oral tetrathiomolybdate (as ammonium or other salt as an immediate release or preferably in sustained release formulation) is given for between 14 days and 1 hour prior and including time 0 on day 0 or simultaneously at time 0 on day 0, (e) the recommended daily allowance (RDA) or greater of any or all of the following essential trace metals, zinc, copper, iron, calcium, molybdenum, and magnesium.

Results:

The administration of the formulations described in (a) through (e) of the preceding example have the effect of lowering the peak and AUC of free copper in the serum samples (and CSF) of all groups of patients.

In the prior art, Bayer T A describes the use of copper supplements for the treatment of Alzheimer's disease based upon the premise that levels of available copper in the CSF and/or CSF of Alzheimer's patients are deficient for normal neuronal metabolic function. Kessler H, Pajonk F G, Supprian T, Falkai P, Muthaup G, Bayer T A, The role of copper in the pathophysiology of Alzheimer's disease, Nervenarzt, 2005 May; 76(5) 581-5 and Bayer T A, Multhaup G, Involvement of amyloid beta precursor protein (AbetaPP) modulated copper homeostasis in Alzheimer's disease, J. Alzheimers Dis. 2005 November; 8(2);201-6; discussion 209-215. The results of the examples above could be reproduced by substituting a Cu⁶⁴ supplement as described by Bayer with Cu⁶⁴ drinking water previously described. Since the immediate release formulation described by Bayer would also result in elevated peak and AUC levels of free copper in the systemic circulation and CSF patients. While an intraday period of neuronal copper deficiency may indeed be the case in Alzheimer's disease, such prior art fails to identify the substantial intraday fluctuations of peak and AUC free copper levels and corresponding temporary compensating overproduction by the brain of APP and Aβ during peak periods as the primary cause of neuronal copper deficiency following hepatic incorporation and biliary excretion of peak free copper levels attributable to the first pass effect of bolus free copper administration. The sustained release bound copper, zinc iron and other trace metal formulations described herein have the benefit of addressing adequate copper, iron and trace metal intake and thereby substantially reduce the neuronal burden to produce the compensating metalloproteins such as APP, Aβ, tau, BACE1 and apoE.

### EXAMPLE 3

### Immediate Release Versus Sustained Release Copper Supplementation

One cohort of 24 Alzheimer's patients are treated once a day for one to three months with an immediate release copper supplement as described by Bayer T A containing 2 mg of copper. A second cohort of 24 Alzheimer's patients are treated with once a day for the same period with a sustained release formulation containing the same quantity of copper either also as a salt or preferably bound to a natural copper binding carrier such as, metallotheionein, fiber, whey or casein. All patients abstain from copper containing drinking water during the study period and efforts are made to balance the groups based upon approximate daily intake of dietary copper-containing foods as well as use of cholesterol lowering agents and other medications. Serial serum samples are taken at least 12 hours away from food every week at points alternately within 1-3 hours following daily dose administration as well as 12 hours away from administration and within two hours of and levels of free copper (using the direct free copper methodologies preciously described herein to measure free copper), total copper, ceruloplasmin and 24S-hydroxy-cholesterol (a copper-implicated oxysterol believed to be produced in the brain that is elevated in the serum of Alzheimer's patients). Results: Peak and calculated approximated AUC levels of free copper in the serum of Alzheimer's patients are increased in the immediate release cohort compared to the sustained release cohort, serum levels of total copper and ceruloplasmin are not statistically significant. Most importantly, however, average time weighted levels of 24S-hyrdocy-cholesterol are significantly higher in the immediate release cohort as compared to the sustained release cohort, indicative of greater oxidative disease process in the CNS in the immediate release group. A crossover design further substantiates the effect.

### EXAMPLE 4

### Gastroretentive Sustained Release Zinc

A double blind placebo controlled clinical trial in Alzheimer's patients is designed comparing placebo (Cohort I), 2 mg/day immediate release copper supplementation (Cohort II), 100 mg/day gastroretentive sustained release zinc acetate without copper supplementation (Cohort III) and 100 mg/day gastroretentive sustained release zinc acetate with 2 mg/day sustained release copper supplementation (Cohort IV) is carried out for 12 to 24 months. Patients are not restricted from consuming normal tap water or bottled water but attempts are made to balance the groups based upon normal habits as well as commonly used concominent medications such as statins (such as atorvastatin), cholinesterase inhibitors (such as donepezil)and NMDA receptor antagonists (such as memantine). The primary endpoint of the study is clinical improvement based upon mini-mental state exam (MMSE) scoring, brain atrophy as measured by volumetric MRI at a minimum of 1.5 T resolution using commonly decribed procedures and longitudinal proton magnetic resonance spectroscopy 1H-MRS utilizing the PRESS-J and autorepositioning techniques described by Hancu I, et. al (2005). Results: Cohort III shows least declined improved but shows a higher incidence of hypocupremia compared to Cohort IV that is also less declined than Cohort I or Cohort II, with Cohort II showing the highest decline in the primary and secondary endpoints described.

Low Dose Tetrathiomolybdate

An object of the present invention are pharmaceutical formulations of low dose tetrathiomolybate useful for the treatment of a variety of diseases. To the knowledge of the inventor, doses of tetrathiomolybdate of less than approximately 0.25 mg/kg have not been previous have not been previously tested or utilized in precious animal model or human disease. The utility of such low doses as contemplated hereby represents a significant departure from the manner in which such agent has previously been utilized. In prior experiments and clinical settings, doses of approximately 1 mg/kg/day to 1.5 mg/kg/day are generally utilized bring the levels of available free copper down to negligible amounts. Such doses are then generally titrated in order to maintain available free copper levels (generally measured indirectly based upon systemic levels of ceruloplasmin since copper bound in a tripartite complex with tetrathiomolybdate and albumin are difficulty to distinguish using currently available practical means) within a certain therapeutic range.

Based upon the observations described herein regarding the substantial intraday fluctuations of peak free copper levels (as measured by the direct measurement techniques described above), an object of the present invention is to provide a low but steady concentration of tetrathiomolybdate in the serum to reduce and stabilize free copper levels in the serum through the binding of free originating from copper free copper fluxes(such as those arising from drinking tap water containing soluble copper) solublized copper in tap water), forming a tripartite complex with serum albumin and thus rendering it unavailable to low molecular weight proteins such as homocysteine, capable of crossing through the blood brain barrier. Copper bound to thiomolybdates and albumin are generally too heavy to cross the blood brain barrier, giving copper complexing agents such as tetrathiomolybdate unique pharmacologic advantages as opposed to copper chelators such as d-penicillamine, trientine, cliquinol, EDTA and other metal complexing agents since free copper loosely bound to low molecular weight proteins such as homocysteine easily cross the blood brain barrier creating a burden on the intraneuronal and extraneuronal cuproprotein mechanisms necessary to maintain appropriate levels of copper homeostasis. In the case of Alzheimer's disease, by stabilizing levels of free copper in the systemic circulation low dose sustained release formulations of tetrathiomolybdate have the important therapeutic advantage of minimizing the upregulation and production of regulatory copper binding proteins such as APP, Aβ and tau. This unique mechanism of action and important advantage is not enjoyed by copper chelators since copper chelators reversibly bind copper from various compartments. Nor has such mechanism of action been appreciated by the prior art which generally are misguided in their approach as exemplified by the often cited pursuit of copper binding agents capable of crossing the blood brain barrier. Such agents, such as trientine and cliquinol, would be expected to be of little or no benefit with regard to stabilizing free copper levels in the CNS. In neurological Wilson's disease, for example, the present inventors have demonstrated that agents such as d-pencillamine and trientine actually serve to also increase the levels of free copper in the CNS due to their nonselective chelation of copper from non-CNS organ compartments such as the liver. In the case of Parkinson's disease low dose thiomolybdates such as tetrathiomolybdate will serve to stabilize and reduce free copper levels in the CNS which have reported to be elevated in Parkisnon's patients as well as downregulate production of the copper and iron binding protein α-synuclein which comprises the majority of Lewy Bodies associated with such disease as well as other synucleinopathies. This benefit will be further enhanced if given in conjunction with iron complexing agents and/or sustained release iron supplements that will serve to induce protective metalloproteins such as transferrin in the intestines and liver and thereby also protect from free iron influxes attributable top soluble iron in tap water and blous free iron concentratuions attributable to immediate release iron supplement formulations. In ALS, the burden placed upon the copper scavanger Cu/Zn superoxidedismutase (SOD1) will also be reduced, which in the case of the familial ALS is genetically deficient in its copper binding properties. The same is also true of the copper binding apoliprotein E, which in variant 4 (apoE4) is devoid of two copper binding cysteine residues that limit the ability of apoE to clear copper from the CNS via uptake by the liver.

Given the important observations of the present invention regarding the therapeutic goal of stabilizing systemic and CSF levels of free copper, one skilled in the art could develop other novel formulations of various agents, such as depo injection formulations of copper binding agents bound to various proteins, pegalated peptides containing cysteine residues for example, pegalated SOD1, and liposomal complexed copper and iron binding agents capable of accomplishing the same result as the free copper resucing and stabilizing mechanism of action of tetrathiomolybdate and more particularly sustained release tetrathiomolybadte and more particularly low dose sustained release tetrathiomolybdate. Accordingly, it is an object of the present invention claim the method of using any agent capable of binding and rendering unavailable free copper wherein such agent is made available in the serum at a steady state and preferably does not cross the blood brain barrier. In one aspect an object of the present invention is to provide stable immediate release pharmaceutical formulations containing thiomolybdates that are useful for chelating and complexing copper in the stomach and gastrointestinal tract and serum. The pharmaceutical formulations and methods described herein are useful to block copper absorption from the intestine while complexing the copper in serum to direct its excretion from the body. Specifically, pharmaceutical formulations are described for complexing and sequestering the copper in food and liquids, and from endogenous sources, such as saliva, gastric, pancreatic, biliary secretions and sloughed enterocytes, when taken with meals thereby reducing copper absorption as well as having use in complexing and reducing the levels of free copper in serum when taken away from food. The present inventions overcomes the conflicting goals of providing an immediate release formulation of thiomolybdates capable of rapidly dispersing in the stomach so as to complex and sequester copper contained in foodstuffs and liquids when taken with meals while at the same time overcoming the inherent instability associated with thiomolybdates so as to provide a pharmaceutical product having a commercially and regulatory acceptable shelf life without compromising the rapid dissolution characteristics necessary to adequately disperse thiomolybdates to complex and sequester copper in the stomach and intestines when taken with meals. The present invention also comprises stable immediate release formulations and methods that when taken away from food are capable of crossing the gastrointestinal digestive tract and chelating and sequestering copper in the serum of a patient in need thereof. The present formulations reduce the frequency of dosing while maintaining adequate whole body copper reduction therapy.

Thiomolybdates:

Thiomolybdates are comprised of molybdenum and sulfur, and include but are not limited to species such as (MoS₄)₂ and (MoO₂S₂)₂. The thiomolybdates can be made as a pharmaceutical acceptable salts, such as, the diammonium salt. These molecules can act as bidentate ligands, and can complex copper. Examples of thiomolybdates include but are not limited to tetrathiomolybdate, trithiomolybdate, dithiomolybdate, and monothiomolybdate. Other examples include complex thiomolybdates, which include but are not limited to a zinc or an iron between two thiomolybdate groups, and which contain thiomolybdate capable of binding or complexing copper. In exemplary complex thiomolybdates, the molecule may have more than four thio groups related to more than one molybdenum. The Group VI transitional metals, tungstate, can substitute for molybdate in formulations, as thio-tungstates.

A thiomolybdate that has particular relevance for the treatment of neurologically-presenting Wilson's disease is tetrathiomolybdate. Tetrathiomolybdate is a thiomolybdate that comprises a molybdenum atom surrounded by four sulfurs, (MoS₄)₂.

Since the 1980's, oral tetrathiomolydate has been the subject of preclinical and human clinical trials conducted by George Brewer, M.D., Fred Askari, M.D. and others for the treatment of initially-presenting neurologic Wilson's disease as well as other diseases that may benefit from the inhibition of intestinal copper absorption and the sequestration and removal of endogenous copper from the body.

Neurologic Wilson's disease is a genetic disease caused by mutations of the ATP7B gene and is characterized by an impaired hepatic ability to incorporate copper into the ceruloplasmin protein and excrete copper yia the bile and stool. This impairment results in elevated levels hepatic and brain copper and that of free copper in the systemic circulation of Wilson's patients which as a result causes toxicities to the liver, brain and other organs.

The regimen of tetrathiomolybdate used for initially presenting Wilson's patients involves up to a sixteen week treatment period in which the patient is sometimes simultaneously administerd Zinc, such as zinc acetate. Treatment can also be followed by daily maintenance therapy with zinc containing agents such as zinc acetate (Galzin®). During the up to sixteen week treatment period with tetrathiomolybdate, tetrathiomolybdate is orally administered to patients up to three times daily with food and with additional administration of tetrathiomolybdate given orally away from food. The goal of tetrathiomolybdate administration with food is to act in the stomach and intestine to complex and render unavailable for systemic absorption copper contained in meals, liquids and endogenous copper that enters the gastrointestinal tract. Tetrathiomolybdate is given away from meals to facilitate its systemic absorption thereby allowing it to form a tripartite complex of toxic free copper in serum to serum proteins, such as albumin, and thereby reduce the availability and levels of toxic free copper levels in the serum and its availability to the central nervous system (CNS). Reduced availability of copper to the CNS can reduce levels of free copper to normal levels and can prevent or treat psychiatric and neurodenerative toxicities of elevated free copper in the CNS.

Tetrathiohiomolydates, under typical atmospheric condition of temperature and humidity are unstable and the active ingredient can undergo oxidation to molybdenum sulfoxide. Molydbdenum sulfoxide is not active as a copper binding agent and therapeutic agent for reduing body copper levels in humans. Tetrathiomolybdates can be expected to lose approximately 10% of its potency within three months if stored under such conditions.

Accomplishing the dual goal of creating an oral thiomolydate pharmaceutical product capable of immediately releasing and rapidly dispersing in the stomach to complex copper contained in meals and liquids while at the same time creating a pharmaceutical product having acceptable stability, shelf life and consistent potency has been the subject of considerable efforts by others in the field. For example, see: U.S. patent application Ser. No. 11/256,540, filed Oct. 21, 2005 by Ternansky et. al which describes solid dosage formulations of thiomolybdate compounds that directly incorporate thiomolybdate in matrix materials (comparable to a lozenge) in order to improve their stability. Such approach has the significant drawback of delaying the release and dispersion of thiomolydates in the stomach and thereby impairing the desired copper complexing effects of thiomolydates in the stomach. U.S. patent application Ser. No. 10/447,585, filed May 28, 2003, describes thiomolybdate analogs having pharmaceutically acceptable salts and esters other than ammonia. While such analogues may have some improved stability as compared to ammonium tetrathiomolybdate, such analogues do not fully overcome stability issues when challenged with moisture or atmosphere and provide no advantage over ammonium tetrathiomolybdate, the agent having the most clinical experience in Wilson's disease.

The current invention in one aspect comprises immediate release capsules containing a thiomolydate wherein the capsule has a very low water content, wherein the capsule contains an exipient that has a low water content and wherein the capsules are packaged in containers or blister packs, to protect the thiomolybdate from the atmospheric oxygen by purging and sealing the capsules with an inert gas, such as nitrogen or argon. The packaging of the capsules may contain one or more chemical indicators to alert the use of a breach of the anhydrous or anaerobic seal of the package with an indicator, such as one for moisture or oxygen.

The present invention also provides a method of timing the administration of a thiomolybdate immediately prior to eating and co-administration of an adequate volume of a liquid to assure the rapid dissolution and the broadest complexation coverage of copper containing foods and liquids.

The present invention also includes the use of low dose sustained release tetrathiomolydate or any other comparable agent as so described to treat other non-CNS disorders in which oxidation due to elevated peak and AUC free copper are known to play a role, such as atherosclerosis and liver diseases such as NASH, non-viral hepatitis and diabetes.

So as to maximize the bioavailability of sustained release formulations of intact tetrathiomolybdate as described herein, sustained release formulations may preferentially comprise two or more microparticle or matrix types contained in a single pill capsule or tablet for example. Such pill or capsule may contain an immediate release form, while one enteric coated particle is designed to release in pH environment of the jejunum and small intestine for example and yet another designed to release in environment of the ileum and colon. Such formulations and methods are known in the art but given that the mechanism of action of tetrathiomolybdate depends upon oxidation of its sulfur groups for its copper binding and albumin complexing properties (and thus its susceptibility to premature oxidation), such formulations will have the advantage of limiting exposure of the tetrathiomolybdate contained within from potential oxidation prior to its release.

To further limit oxidation and improve stability of tetrathiomolybdate, the present invention also comprises a pharmaceutical package whereby each pill, tablet or capsule is separately sealed in its own modified atmosphere packaging such as an impervious foil pouch or cold form blister pack that is purged under argon or nitrogen gas to expel 98% or greater of atmospheric air and moisture until ready for use. If capsulues are utilized, capsules containing tetrathiomolybdate should also be specifically selected for low moisture content so to limit interaction between the capsule and active ingredient. Pouches or blister pack packaging can be further packaged in an inert purged package together with an desiccant and sacrificial oxidant to improve stability.

In a particularly preferred embodiment of the invention, the size of the tetrathiomolybdate crystals are controlled to provide selected, sustained or delayed release in the stomach and the intestines. Crystal size may be controlled through controlled crystal growth, or by milling as described below. We have found that larger tetrathiomolybdate crystals, for example, ammonia tetrathiomolybdate pass through the stomach essentially unchanged and dissolve in the gut, while smaller crystals dissolve in the stomach. Thus, sustained release in relation can be achieved by providing a mixture of crystals of varying sizes. For example, a mixture comprising crystals of 50-100 microns and 200-500 microns may be provided, in a single capsule.

We have found that the kinetics of ammonium tetrathiomolybdate (ATTM) dissolution in artificial gastric fluid is dependent on the size of ATTM crystals. ATTM crystals that were 50 microns or less (width cross-section) in size dissolved rapidly in acidic buffer (pH 2). After 1 hour of incubation in acidic buffer no ATTM was found. However, ATTM crystals that were 1700 microns or greater in size dissolved slowly in acidic buffer. After 1 hour of incubation in acidic buffer, more than 50% of ATTM was found.

Method:

1. ATTM purchased from Sigma-Aldrich was sieved using a 1700 micron screen. Crystals which did not pass through the screen were called large and used in the experiment.
2. ATTM crystals were ground so that no crystals were larger than 50 microns. These will be referred to as small crystals.
3. 100 mg of each type of crystal was weighed and added to separate 1.7 ml plastic centrifuge tubes.
4. 1 ml of buffer pH 2 was added to each tube without mixing.
5. After 1 hour, each tube was centrifuged at 14,000 rpms for 1 minute.
6. The supernatant was removed and analyzed for ATTM content.
7. To the pellet of each tube was added 1 ml of phosphate buffer pH 7.4
8. Each solution was vortex-mixed.
9. Each tube was centrifuged at 14,000 rpms for 1 min.
10. The supernatant from each tube was analyzed for ATTM content.
   Analysis:
1. A 1 to 100 dilution of all supernatants were made in phosphate buffer pH 7.4.
2. 2 ml of each diluted sample were added to plastic cuvettes.
3. Absorbance was measured at 467 nm.
4. The extinction coefficients (EC) of ATTM for each sample were calculated.

Results:

| Sample | Absorbance at 467 | Extinction Coefficient |
|---|---|---|
| Small Crystal Supernatant | 0 | 0 |
| Small Crystal Pellet | 0 | 0 |
| Large Crystal Supernatant | 0 | 0 |
| Large Crystal Pellet | 0.310 | 7,000 |
| Theory | 0.600 | 13,500 |

Observations:
1. When acid buffer was added to small crystals of ATTM, the solution turned immediately black and a sulfur odor could be detected.
2. The black solution eventually resulted in a black precipitate that settled to the bottom of the tube leaving the supernatant clear. This was observed by the 1 hour incubation time point.
3. When acid buffer was added to large crystals of ATTM, a small amount of black coloration leached from the crystal. In addition, a black coating was found to form on the exterior of the crystals which seemed to resist further dissolution.
   Conclusions:
1. As small crystals, ATTM dissolves in Acid media but soon forms an insoluble precipitate.
2. Dissolved ATTM does not survive in acid media.
3. In acid, as large crystals, ATTM forms a black coating which seems to resist dissolution.
4. In acid, un-dissolved ATTM from large crystals can be liberated with a secondary dissolution in neutral media.
5. The black coating does not appear to prevent dissolution in neutral media.

### EXAMPLE 5

Large (>1000 microns) and small (<50 microns) dark orange crystals of tetrathiomolybdate are placed in a solution of a commercially available simulated stomach acid. The large crystals release a slight odor of sulfur, the crystals remain relatively intact, turn black, and the solution remains clear. The small crystals are placed in the same type of solution and immediately release a strong odor of sulfur and form a disaggregate cloudy black solution. The dissolution properties of the large verses small crystals are clearly distinct. To further examine the properties of a methylcellulose capsule containing 20 mg of tetrathiomolybdate and 180 mg of excipient containing a heterogenous size range (200-800 microns) of tetrathiomolybdate crystals was examined in a dissolution chamber initially containing simulated stomach acid. The gelatin capsule dissolved within 15 minutes. Tetrathiothiomolybdate recovery was assessed by UV/Visible absorption periodically over 3 hours, a time consistent with the initiation of gastric emptying and the initial phase of postprandial absorption. Tetrathiomolybdate was not detectable in the simulated gastric samples removed and neutralized at any time point over this three hour period, suggesting the agent did not dissolve or was disaggregated. At three hours the remaining gastric juice was made alkaline by adjusting the pH to 8-9, the solution turned orange to red and was assessed by UV/V is spectroscopy to determine the recover of tetrathiothiomolybdate, which was approximately 7% of the amount in the capsule.

When the same experiment is performed with small crystals, much less than 7% of parent compound is recovered, while when the experiment is performed with large crystals much greater than 7% of parent compound is recovered. Overall these experiments demonstrate that tetrathiothiomolybdate crystal size is a determining factor for protection from degradation by stomach acid.

### EXAMPLE 6

Tetrathiothiomolybdate crystals are either crushed to a powder, that is initially orange in color, or placed in water to dissolve yielding a bright orange solution, which is filtered and lyophilized, which results in a dry powder orange in color. The crushed crystals are purged with nitrogen overlay and slowly turn black within a few day at room temperature in a closed tube, while the lyophilized dry powder under similar conditions remains orange for over a month. This experiment suggests the thorough removal of water from tetrathiothiomolybdate improves stability to the atmosphere. Similarly, when orange tetrathiomolybdate is protected from the atmosphere and moisture by covering with mineral it remains orange (i.e. not oxidized) for over a month.

### EXAMPLE 7

Similar amounts of lyophilized tetratiomolydate powder is dissolved in water, placed in a capsule, prepared as a non-coated tablet, or enteric coated tablet and dosed once a day in fed or fasted rats for one week. At the end of the multiple dosing period, blood samples are taken periodically over 24 hours for pharmacodynamic determination of ceruloplasmin and pharmacokinetic determination of molybdenumum. In both the fasted and fed animals, ceruloplasmin was reduced to the greatest extent in animals administered the enteric coated tablets and these animals also had the greatest increase in serum molybdenumum.

### EXAMPLE 8

### Low Dose Tetrathiomolybdate

A double blind placebo controlled clinical trial in Alzheimer's patients is designed comparing placebo (Cohort I), to low dose (0.2-10 mg/day) sustained release ammonium tetrathiomolybdate (Cohort II) is carried out for up to 24 months. The primary endpoint of the study is clinical improvement based upon cognitive assessments such as mini-mental state exam (MMSE) scoring, brain atrophy as measured by volumetric MRI at a minimum of 1.5 T resolution utilizing commonly described procedures and longitudinal proton magnetic resonance spectroscopy 1H-MRS utilizing the PRESS-J and autorepositioning techniques described by Hancu I, et. al (2005). Results: Cohort I shows least decline in all parameters as well as a low incidence of hypocupremia compared to Cohort II.

### EXAMPLE 9

### Tetrathiomolybdate Followed by Gastroretentive Sustained Release Maintenance Therapy

A double blind placebo controlled clinical trial in Alzheimer's patients is designed comparing placebo (Cohort I), to an induction dose of 80-120 mg/day of immediate release ammonium tetrathiomolybdate for three to six months (as described in the inventor's U.S. Pat. No. 6,855,340 and U.S. patent application Ser. No. 10/444,204) followed by a daily maintenance therapy of 50-100 mg/day of gastroretentive sustained release zinc (with or without sustained release copper supplementation as needed as determined by levels of systemic ceruloplasmin levels of less than a targeted level such as below 12 mg/dl and/or clinical indications of hypocupremia) for an additional 18-21 months. The primary endpoint of the study is clinical improvement based upon cognition which can be measured with mini-mental state exam (MMSE) scoring, brain atrophy as measured by volumetric MRI at a minimum of 1.5 T resolution utilizing commonly described procedures and longitudinal proton magnetic resonance spectroscopy 1H-MRS utilizing the PRESS-J and autorepositioning techniques described by Hancu I, et. al (2005). Results: Cohort II shows less decline in all parameters as compared to Cohort I and potential objective indications improved metabolic brain function in as little as 3-6 months as compared to Cohort I.

Molybdenum and Sulfur Containing Formulations

An object of the present invention includes oral nutriceutical formulations that contain elemental molybednum and sulphur. Upon administration and dissolution in the stomach such formulations would combine in the low pH environment of the stomach and form thiomolybdates capable of complexing free copper in the gastrointestinal tract and systemic circulation much like ammonium tetrathiomolybdate. While such formulations would be expected to be far less reliable in terms of dosing, they might have the advantages as natural products purusnat to the Dietary Health Supplement and Education Act (DSHEA).

While it is possible that, for use in therapy, a sustained release trace metal of the invention may be incorporated into sustained release pharmaceutically acceptable sustained release microsphere, matrix, pellet or particle (all of which are commonly known in the art) in the form as a pure cation or salt, it is preferable to first bind the trace metal to a pharmaceutically acceptable, stable, natural or synthetic carriers to which such metals are known to bind, such as, for example, plant fiber, whey, metallotheionein, transferrin, proteins and/or milk or milk by-products. Such carriers will have the benefit of further inducing the gradual digestion and absorption of the trace metals as they are naturally found in foods. The invention thus further provides a gastroretentive and/or sustained release pharmaceutical formulation incorporating one or more trace metals, such as zinc, copper and iron together with one or more pharmaceutically acceptable carriers and, optionally, other therapeutic and/or prophylactic ingredients. The carrier(s) must be “acceptable” in the sense of being compatible with the other ingredients of the formulation and not deleterious to the recipient thereof.

Another object of the present invention is to provide a copper supplement that may be administered in conjunction with, or combined with, a copper malabsorption agent, wherein the copper is in a form bound to or formulated with lipids, whey or casein. A copper supplement formulated in such a fashion would be intended to mimic, in pill, capsule or liquid form, the manner in which copper is provided from mother to infant via human breast milk. It would therefore be expected that such copper, upon reaching the stomach and small intestines, would be appropriately processed in a proper digestive manner, as opposed to copper which is in water or in a pure salt form. It is anticipated that the processing of such a copper supplement would mimic the high bioavailability found in breast milk (24%) or cow milk (18%), while also permitting the normal digestion and processing of copper by the intestines and liver, thereby reducing the level of burden of free or loosely bound copper in the serum and CNS. Such formulations could also include other essential metals and minerals such as iron or zinc. In a preferred embodiment of the invention, a copper and/or iron supplement formulated with lipids, whey and other proteins, with which copper is normally found in breast milk, may be formulated with a copper malabsorption agent such as zinc so as to simultaneously provide a bioavailable amount of copper and/or iron in a form for normal processing by the intestines, while at the same time inducing the production of metallothionein in the intestines to block and protect against the subsequent absorption of ionic copper from drinking water. In a preferred embodiment, such carrier bound copper supplement is incorporated within a sustained release microparticle or matrix so as to further regulate the absorption, and reduce the potential to cause peak elevated levels of free copper in the systemic circulation and CSF. Such formulations should reduce or avoid the need to monitor patients for hypocupremia or anemia, and also lower the levels of free or loosely bound serum or CNS copper while bolstering the levels of ceruloplasmin bound copper (given its processing by the liver by virtue of the first pass effect and normal copper handling, which mimics that of the evolutionarily proven copper and metal supplementation methods by which a mother processes and passes nutritional copper and other metals to a newborn baby via breast milk). Such formulation could also include other essential trace elements in a carrier bound complexed sustained release formulation, such as, iron, calcium, molybdenum, selenium, and magnesium, for example. Complexed sustained release calcium formulations are useful for the treatment or prevention of diseases involving calcification such as in the arteries, kidneys, lungs and brain.

In a preferred embodiment of the invention, stable copper isotopes Cu⁶⁵ and Cu⁶⁷ are utilized as the active copper ingredient to permit copper balance and treatment effect to be evaluated and adjusted on an individual basis. Such studies may be conducted by sampling serum, CSF fluid, stool, or urine, or by biopsy. Such information provides a useful diagnostic method to evaluate the effects of other therapeutic approaches, such as therapies to improve hepatic excretion of copper as well as copper chelators.

The zinc and copper formulations described herein may be administered as a nutritional supplement, parenterally, by immediate release, or by depo release injection. Oral nutritional formulations of zinc and complexed copper may also contain certain copper absorption enhancing agents such as glycerol and NaCl, or gum arabic, to increase the bioavailability of complexed copper.

The copper and zinc formulations described herein may also be useful for the treatment of gastrointestinal disorders such as chronic diarrhea, diarrhea predominant irritable bowel syndrome and infections for which zinc is known to be beneficial.

The present invention may be administered as a two-pill system whereby complexed copper pills or formulations are orally administered first and are followed by orally administered zinc-containing pills either together or after sufficient delay.

The present invention also contemplates incorporation of other essential minerals for which intestinal zinc may also reduce bioavailability, such as iron and cadmium.

The present invention may be preferentially combined with a cholesterol lowering agent for the treatment or prevention of atherosclerosis, dementia, Alzheimer's disease and other neurogenerative diseases, including neuromuscular diseases, which are associated with abnormal accumulation of copper associated proteins, such as juvenile and sporadic inclusion body myositis and myositis of the elderly.

A further embodiment of the present invention contemplates the use of the zinc formulations described herein or copper lowering agents for the treatment of neurologic and psychiatric manifestations of hepatic diseases associated with impaired liver copper excretion, such as colangitis, hepatitis and cirrhosis, for example, in which free or loosely bound serum or CSF copper is elevated.

### EXAMPLE 10

A pill or capsule containing high-density 100 mg zinc salt pellets capable of retention in the walls of the stomach is administered to groups of individuals in the morning. A control group of individuals receives a 100 mg capsule of normal zinc acetate, while another control group receives a placebo. Such individuals consume copper-containing or Cu⁶⁴ containing distilled or tap water at identical times throughout the day. Serum samples are obtained before, during, and after each drinking event. Such samples are immediately measured for their respective free or loosely bound copper content utilizing total serum copper less copper bound to ceruloplasmin, direct measurement copper bound to various known and unknown proteins and peptides by means of separation based upon molecular weight, as per the column method described by Bohrer D (2004) or MALDI-TOF described by Sarkar E (2004).

Results: The placebo group shows the highest peak levels of free or loosely bound copper, the bolus oral zinc group has a lower peak free or loosely bound copper level, while the gastroretentive/sustained release zinc group shows the lowest levels of free or loosely bound free copper. To demonstrate long-term benefits of intestinal metallothionein induction to block copper absorption, the experiments are repeated daily until statistical significance is achieved.

### EXAMPLE 11

Copper, copper salts or copper bound to low molecular weight amino acids is dissolved in a solution of dried or evaporated milk, dried whey, dried milk lipids, or dried milk proteins or other natural copper binding proteins. The resulting mixture of copper bound complexes may be dried and formulated as a pill or tablet. In a preferred embodiment of the invention, the dried copper bound complexes are formulated in a pill or capsule together with zinc. In another preferred embodiment, the pill or tablet is formulated utilizing gastrorententive forms of zinc, enteric coated zinc and/or sustained release zinc such that the copper bound complexes are released into the gastrointestinal tract ahead of the zinc. The copper bound complexes are digested in the GI and absorbed by enterocytes where they are processed intracellularly with metallothionein and/or transcuprein, whereupon such metallothionein and/or transcuprien bound copper enters the liver and is taken up for processing and incorporation into ceruloplasmin and then either released into the serum, retained in the liver or excreted into the bile. Subsequently, the later passing zinc is absorbed in the enterocytes, thereby upregulating metallothionein, which serves to block subsequent absorption of solubilized copper (free or loosely bound copper such as copper-containing tap water or other liquids that would otherwise pass into the hepatic circulation upon water flux without proper processing by the enterocyte and enter the hepatic circulation bound to albumin). As a result, a substantial portion of such portal free or copper loosely bound to albumin will enter the liver and subsequently enter the serum circulation in a similar free or loosely bound form, resulting in oxidation and other toxicities to arterial, neuronal and other organ systems of the patient, thereby promoting atherosclerosis, dementia, mild cognitive impairment, Alzheimer's disease, Parkinson's disease and other diseases associated with elevated levels of free copper.

### EXAMPLE 12

Animals or humans are orally administered short half life radioactive isotopes Cu⁶⁴ or Cu⁶⁷ and/or the stable copper isotopes Cu⁶³ or Cu⁶⁵ formulated as capsules or pills as described above and also as solubilized in water. A cross-over design is utilized, whereby naturally occurring copper (39% Cu⁶³ and 69% Cu⁶⁵) is administered orally as a pill as described above or solubilized in water. Such administration may be short, or for up to 90 or more days in the case of the stable copper isotopes. Portal and/or serum samples are collected and fractionated into components utilizing columns capable of separating ceruloplasmin, transcuprien, albumin, small proteins and peptides. The copper isotopes bound to each component are measured, demonstrating a statistically significant greater proportion of ceruloplasmin and transcuprien bound copper isotopes administered in the protein bound form as compared to the solubilized form, while the copper isotopes administered solubilized in water show a greater percentage loosely bound to albumin and other low kinetic binding proteins and amino acids. This effect is ameliorated with the pill or capsule formulation which contains early release copper and gastroretentive zinc, as the zinc protects against subsequent absorption of free copper solubilized in water, while reducing the tendency for hypocupremia. The results are more pronounced in animals and patients having impaired liver function such as patients or animal models of cirrhosis, hepatitis, reduced biliary flow, and primary sclerosising oholengiatis as well as geriatric patients. The clinical benefits of this invention may be demonstrated in animal models of atherosclerosis and Alzheimer's disease, such as Taconic rats, by measuring plaque volumes as well as memory tests.

### EXAMPLE 13

### Dissolution of ATTM as a Capsulated Formulation

Introduction:

We performed a dissolution experiment using a capsule containing 20 mg of ammonium tetrathiomolybdate plus excipient. ATTM crystals used were 500 microns or smaller. No ATTM was recovered in the gastric dissolution medium at any time. After, 3 hours of dissolution, black particles released from the capsule were found suspended in solution. After neutralization of the gastric media to pH 9, these black particles disappeared resulting in about 10% of ATTM being recovered.

Summary:

1. No ATTM was found dissolved in study fluid
2. ˜10% of undissolved ATTM remained in study fluid following 3 hours of dissolution.
3. Conclusion: ˜90% of ATTM is destroyed in the stomach.
   Method:
   Material Studied
1. Study was performed on R&D Pipex formulated capsule of ATTM.
2. The ATTM was milled through a 500 micron sieve.
3. The capsule contained ˜20 mg of ATTM and 180 mg of excipient.
   Dissolution Conditions
1. Media: 1 liter of UPS artificial gastric fluid pH 2
2. Temp: 22° C.
3. Paddle RPMs: 100
4. Dissolution time: 3 hours
   Procedure
1. A #2 QualiCaps capsule available from Shionogi Europe, B.V. was mechanically dropped into media at time zero
2. 1 ml of media was then collected at 1, 2, 4, 8, 16, 30, 45, 75, 120 and 180 min
3. Following 3 hours of dissolution, the pH was raised to 9 using NaOH pellets.
4. The media was paddled for 1 hour.
5. After 1 hour a sample was collect for analysis
   Analysis
1. 400 ul of collected media was diluted to 2 ml with phosphate buffer, pH 7
2. The solution was placed in a cuvette and scanned between 260 and 600 nm.
3. Absorbance was measured at 367 nm.
4. The extinction coefficient was calculated using Beer's law.
5. The extinction coefficient of a reference sample in a capsule is 13500 cm⁻¹ M⁻¹
   Conclusion:
1. Any ATTM that dissolves in media at pH 2 is immediately destroyed.
2. Some of the ATTM floated in the media as black specks following 3 hours of dissolution.
3. After three hours when the pH of the solution was raised to pH 9, the color of the solution changed to an orange color.
4. This presumably is stable ATTM that has dissolved.
5. The amount of ATTM remaining was only about 10% of theory.
6. Therefore 90% of the original dose of ATTM was destroyed in the artificial gastric fluid over three hours.
7. With enteric coating, much lower doses may be required to achieve the same therapeutic effect.

### EXAMPLE 14

### Stability of ATTM Capsules after Eight Weeks

Introduction:

Ammonium tetrathiomolybdate formulated capsules were studied for stability. Various conditions were examined. The best formulation was found when QualiCaps capsules (low moisture methylcellulose) were used, nitrogen was used as the storage atmosphere and the excipient included lactose monohydrate.

Summary:

1. ATTM was completely stable after 8-Weeks when stored at RT and ambient RH.
2. ATTM was 60% to 90% stable after 8-Weeks when stored at 40° C. and 70% RH.
   Formulation performance at 40° C. and 70% RH:
1. QualiCaps capsules better than gel caps (4 of 4 cases)
2. Nitrogen better than room air (3 of 4 cases)
3. Lactose monohydrate better than lactose anhydrous (3 of 4 cases)
   Conclusion:
1. The best formulation was QualiCaps capsules, nitrogen and lactose monohydrate
2. The worst formulation was gel caps, room air, and lactose anhydrous.

Various procedures are possible for both preparing the ammonium tetrathiomolybdate (ATTM) or ammonium polysacharide (API), preparing formulations and capsules containing same, and the use of same including:

Milling Ammonuim Tetrathiomolybdate (ATTM)

1. A method for milling ATTM under condition of low moisture and oxygen levels that separates ATTM from oxidized forms of the active ingredient that present as black tar-like by-products (flaky tar).
2. A method of 1 whereby granulation of ATTM produces powdered ATTM of reduced average particle size allowing separation from flaky tar-like by products.
3. A method of 1 whereby ATTM is sieved to produce powdered ATTM and flaky tar.
4. A method of 1 whereby sieves are selected to separate granular and powdered ATTM from flaky tar.
5. A method of 1 whereby powdered ATTM is separated from flaky tar in a container in which vibration is used to separate fine ATTM particles from coarse flaky tar.
6. A method of 1 whereby mechanical vibration and gravity allows fine ATTM particles to separate and collect below coarse flaky tar.
7. A method of 6 whereby layers of coarse flaky tar are removed from layers of fine ATTM particles.
8. A method for milling ATTM so that powdered ATTM that has been separated from tar is milled into ATTM particles of average reduced size under conditions of low moisture and oxygen.
9. A method of 2 whereby ATTM of reduced average particle size dissolves rapidly in an aqueous solvent or the environment of the gastrointestinal tract.
10. A method of 1 by which the granulation of ATTM is milled using employing a mechanical device, mechanical (such as a hand sieve) or power driven (such as a blender) that produces particles of reduced average particle size compared to the starting ATTM.
11. A method of 10 where the milling process produces an average particle size that is less than 100 microns.
12. A method of 10 where the milling process produces an average particle size that is less than 10 microns.
13. A method of 10 where the milling process produces an average particle size that is less than 1 microns.
   Excipient Selection
14. A method of formulation whereby the ammonium polysacharide (API) is mixed with an excipient that enhances the pharmaceutical properties of the API.
15. A formulation of 14 in which the excipient limits the moisture content of API.
16. An example of 15 but not limited to the example is anhydrous lactose.
17. A formulation of 14 in which the excipient limits the oxidation of API.
18. An example of 17 but not limited to the example is a crown ether.
19. A formulation of 14 in which the excipient limits the reaction of reactive oxygen and/or nitrogen species with the API.
20. An example of 19, that contains one or more free radical scavengers or antioxidants, such as but not limited to, mannose, and butylated hydroxytoluene.
   Salt Selection
21. A method of formulation whereby the API forms a salt with a cation that enhances or protects the pharmaceutical properties of the API.
22. A formulation of 21, in which the cation is an antioxidant.
23. An example of 22 but not limited to the example is the nicotinic ester of tocopherol.
24. A formulation of 21, in which the cation is hydrophobic and acts to prevent the hydration of the API.
25. An example of 24 but not limited to the example is benzylamine.
   Agents Added to the Excipient which Prevent the Acid Hydrolysis of the API
26. A method of formulation whereby an agent as part of the excipient is added to the API to prevent the acid hydrolysis of API.
27. A formulation of 26 in which stomach acid hydrolysis of API is prevented with the addition of an anti-acid to the excipient.
28. A formulation of 26 whereby an anti-acid is administered slightly before, simultaneously, or slightly after, but separately from the ATTM pharmaceutical formulation.
29. An example of 27 but not limited to the example is sodium bicarbonate.
30. An example of 28 but not limited to the example is sodium bicarbonate.
31. A formulation of 26 in which stomach acid hydrolysis of API is prevented by complexation of API to an agent added to the excipient.
32. A formulation of 26 in which upon dissolution of API in stomach fluids, the API complexes with an agent added to the excipient which binds the API and prevents the acid hydrolysis and oxidation of API.
33. An example of 32 but not limited to the example is an aminopolysacharide (API). An example of an aminopolysacharide is polyglucoseamine or chitosan.
34. A mechanism of 33 in which chitosan added to the excipient forms an insoluble colloidal complex with the API following dissolution in stomach acid and thereby protects the API from acid hydrolysis.
35. A formulation in which some or all of the ATTM is enteric coated thereby affording delayed dissolution of active ingredient until the acidic environment of the stomach is neutralized or the ATTM reaches a portion of the gastrointestinal tract that is neutralized.
36. A method of 35 where some or all of the API is enteric coated as microtablets that are encapsulated in a capsule.
37. A method of 35 where the enteric coating is variable to afford variable rates of dissolution of the microtablets.
38. A method of 35 where a single tablet is enteric coated for dosing. 39. A method of 35 whereby tablets of various enteric coatings are prepared for administration to a patient such that the enteric coatings release the API at different rates.
40. A method of 35 whereby an enteric coated formulation is administered with a copper lowering medication that is not enteric coated.
41. A method of 40 where a copper lowering mediation is but not limited to a zinc acetate, zinc sulfate, tetrathiomolybdate, tetrathiotungstate, penecillamine, or trientine.
   Agents Added to the Excipient which Aid in the Absorption of the API.
42. A method of formulation whereby an agent as part of the excipient is added to the API so that a complex is formed which provides for enhancing absorption of the API.
43. An example of 42 but not limited to the example is an aminopolysacharide. An example of an aminopolysacharide is polyglucoseamine or chitosan.
44. A mechanism of 43 in which chitosan added to the excipient forms an insoluble colloidal complex with the API following dissolution in stomach acid and is metabolically converted into di- or tri-saccharides complexes with API that suitable substrates for absorption.
   Material for API/Excipient Capsulation
45. A method of API/excipient capsulation in which capsule materials are selected which enhance the pharmaceutical properties of the API.
46. A capsulation of 45 in which the capsule materials prevent the hydration of API.
47. A capsulation of 45 in which the capsule materials contain very low moisture content.
48. A capsulation of 45 in which the capsule materials are resistant to leakage under acidic conditions.
49. A capsulation of 45 in which the capsule materials are highly soluble in only acid conditions.
50. A capsulation of 45 in which the capsule materials are highly soluble in only neutral conditions.
   Coatings of Capsules or Tablets which Targets Specific Organs for API Dissolution
51. A method of formulation whereby coatings are used to target API dissolution in specific organs of the GI tract.
52. A method of coating in 51 whereby API resists dissolution in the oral cavity but readily dissolves in the stomach.
53. A method of coating in 51 whereby API resists dissolution in the stomach but readily dissolves in the upper and lower intestines.
   Combined Targeting of Specific Organs for API Dissolution
54. A method of formulation whereby capsulation and coatings are used to provide for API release in all organs of the GI tract from a single dose of API.
55. A formulation of 54 which utilizes a combination of release mechanisms.
56. An example of 55 in which API release is targeted for dissolution in the stomach. Dissolution in the stomach is desired for complexation of copper in the stomach from food and drink as a preventative measure in terms of copper absorption occurring later in the intestines.
57. An example of 56 would be finely powdered API and excipient contained in a capsule that readily dissolves in stomach acid. The excipient may contain an API complexing agent which prevents acid hydrolysis but allows for API interaction with free copper in the stomach.
58. A formulation of 55 in which a portion of the API dose is packaged for dissolution in the intestines.
59. A formulation of 55 in which for example a portion of the API dose is coated so that dissolution takes place in the intestines.
60. A formulation of 55 in which a capsule contains powdered API and excipient for dissolution in the stomach. In addition the capsule contains a tablet of coated API for dissolution in the intestines. In this way, API dissolution in the stomach is used to bind copper which prevents copper absorption and API dissolution in the intestines is allowed for systemic API absorption for copper binding systemically.
   Co-Formulation of API with other Natural Products known to Complex Copper
61. A method of formulation in which natural products known to complex copper are added to the excipient.
62. An example of 61 but not limited is sodium alginate. Sodium alginate in powder form is a common food stuff that is known to bind heavy metals and prevent their systemic absorption.
63. A mechanism of 61 would involve the use of sodium alginate to bind free copper from food in the stomach while allowing for coated API to dissolve in the intestine for systemic absorption.
   Formulation of API that Provides for Sustained Release
64. A method of formulation which allows for sustained release of API in the stomach.
65. A formulation of 64 in which API is mixed with an agent that is semi-resistant to dissolution in stomach acid while also possessing an overall material density that provide for buoyancy in stomach fluids.
66. A non-limited example of 65 would be the formulation of API in a digestible wax. The density of the wax would provide for buoyancy in stomach fluids and the slow digestibility of the wax would provide for the sustained release of API.
   Functional Features
67. A method to reduce the number of times a copper reducing therapy is orally administered to a patient.
68. A method in which ATTM is administered to a patient in which the absorption of the API is increased.
69. A method in which a formulation of ATTM is administered to a patient to enhance the pharmacodynamic profile of the agent at an equivalent dose.
70. A method in which a formulation of ATTM achieves indications of serum copper, serum ceruloplasmin, urinary copper, or hepatic copper lowering at a lower dose.
71. A method in which a formulation of ATTM achieves an indication of copper more rapidly than an equivalent dose.
   Monitoring and Dosing
72. A method by which a patient or care giver monitors the effectiveness of an ATTM containing formulation or dose program by assaying a body fluid, excretion or organ for a therapeutic marker indicative of effective copper reduction therapy.
73. A method of 72 where the body fluid is urine.
74. A method of 72 where the body fluid is blood or blood serum.
75. A method of 72 where the body fluid is blood plasma.
76. A method of 72 where the body fluid is saliva.
77. A method of 72 where the body fluid is cerebral spinal fluid.
78. A method of 72 where the material assayed is stool.
79. A method of 72 where the material assayed is liver.
80. A method of 72 where longitudinal imaging methods, such as but not limited to proton magnetic resonance imaging, magnetic resonance imaging, computer topography of the brain or other organs, indicates a slowing of progression or regression of disease.
81. A method of 72 where longitudinal cognitive assessments of a patient are made to indicate a slowing of progression or regression of disease.
82. A method of 72 where quality of life assessments of a patient are made to indicate a slowing of progression or regression of a disease.
   Diseases for Treatment
83. A method of 1 where the formulation is for treatment of disease in need of a copper reduction therapy
84. A method of 84 whereby the formulation is used to treat or prevent a central nervous system disease, an inflammatory disease, a disease in which fibrosis is a component of the disease process, or an angiogenic disease including cancer.
85. A method of 84 where the central nervous disease can be, but is not limited to; Wilson's disease, Alzheimer's disease, Huntington's disease, Schizophrenia, Parkison's Disease, ALS, and Prion diseases.
86. A method of 84 where the inflammatory disease can be, but is not limited to; psorisis, rheumatoid arthritis, lupus, inflammatory bowel disease, etc.
87. A method of 84 where the fibrtotic disease can be, but is not limited to; idiopathic pulmonary fibrosis, primary biliary cirrhosis, nonalcoholic steatohepatits, liver cirrhosis, glomerulonephritis, systemic fibrosis and rheumatoid arthritis, post-surgical adhesions, adult respiratory distress syndrome (ARDS); coal workers' pneumoconiosis (CWP); Hermansky-Pudlak syndrome (HPS); systemic sclerosis (SS), tumour stroma in lung cancer, and obliterative bronchiolitis (OB) after lung transplantation.
88. A method of 1 to treat acetaminophen overdose or poisoning in a mammal.
89. A method of 1 to prevent or treat a copper overdose or poisoning in a mammal
   Formulation of API that Provides for a Protein-API Complex that forms in the Stomach, is Impervious to Acid Hydrolysis, Complexes with Copper Released in the Stomach from Food and is Excreted from the Bowel and not Absorbed into the Blood Stream.
90. A method of formulation in which protein is added as part of the excipient.
91. A method of 54 in which the protein added as part of the excipient forms a stable complex with the API in the stomach.
92. A method of 54 in which the stable API-protein complex is capable of complexing copper in the stomach.
93. A method of 54 in which the stable API-protein-copper complex prevents the absorbtion of API-copper complex into the blood stream once the complex passes beyond the stomach.
94. A method of 54 in which the stable API-protein-copper complex passes through the GI tract and exits with feces.
95. An example of a formuation of 54 would be that bovine serum albumin is added to the excipient.
96. A mechanism of 54 would be that the API and the bovine serum albumin would form a stable complex capable of complexing with copper in the stomach.
97. An example of a formuation of 54 would be a synthetic D-polypeptide which complexes with copper in which the complex can further complex with copper and exit the GI tract intact and is not digested.
98. A mechanism of 61 in which the D-polypeptide is not digested but as a complex with the API and copper exits the GI intact.
   A Method of Preparing API in a Purified Form.
99. A method of preparing purified ATTM using dissolution followed by lyophilization.
100. A method of 99 in which ATTM is dissolved in pure distilled water and purged with inert gas (e.g. nitrogen, argon, etc.)
101. A method of 99 in which ATTM dissolved in water and purged with inert gas is filtered through a sterile 0.22 micron (or less) filter.
102. A method of 99 in which a filter aqueous solution of ATTM is poured into a chilled bottle (e.g. −80° C.) followed by swirling to form a frozen film on the interior surface of the bottle.
103. A method of 99 in which bottle contents is then subjected to lyophilization.
104. A method of 99 in which following lyophilization, the evacuated bottle is filled with pure dry argon or nitrogen.
105. A method of 99 in which ATTM is collected under an inert gas and stored.
   A Method of Tableting Lyophilized ATTM.
106. Lyophilized ATTM is tableted as a neat substance of as a formulation.
107. A method of 106 in which tableted ATTM processes pharmaceutical properties that provide for sustained release.
108. A method of 106 in which tableted ATTM processes pharmaceutical properties that provide for release into targeted organs (e.g. enteric coatings).

While the invention has been described with respect to treating excessive metal buildup or metal maladsorption in animals and humans, other utililties are possible. For example, the use of controlled crystal size to provide selected, sustained or delayed release as described in connection with ATTM advantageously could be used to effect controlled delivery of other active agents.

1. A pharmaceutical composition comprising a sustained release formulation of an agent that induces copper malabsorption in an animal or human.

2. The pharmaceutical composition of claim 1, wherein the composition is formulated to initiate release of the agent in the small intestine or upper jejunum.

3. The composition of claim 2, packaged within a delayed release coating.

4. The pharmaceutical composition of claim 1, wherein the agent is zinc, preferably a zinc-cysteine complex, more preferably zinc-monoysteine. salt.

5. The pharmaceutical composition of claim 1, wherein the agent is a zinc.

6. The pharmaceutical composition of claim 5, wherein the zinc salt is selected from the group of zinc acetate, zinc carbonate, zinc sulfate, zinc gluconate, zinc oxide, zinc chloride and zinc stearate.

7. The pharmaceutical composition of claim 4, wherein the dosage of zinc is between 25 mg and 150 mg.

8. The pharmaceutical composition of claim 4, wherein the zinc is released between 30 minutes and 24 hours.

9. A pharmaceutical composition comprising a sustained release depo injection of zinc, preferably a zinc-cysteine complex.

10. The pharmaceutical composition of claim 9, wherein the zinc is released between 7 and 30 days.

11. A transdermal sustained release patch comprising zinc, preferably a zinc-cysteine complex, wherein the zinc is delivered through the skin.

12. A gel comprising zinc, preferably a zinc-cysteine complex, and an absorption-enhancing excipient capable of delivering zinc through the skin.

13. An underskin implant that administers a continuous release of zinc, preferably a zinc-cysteine complex, for a period of least at least 30 days.

14. An underskin implantable pump that administers a continuous release of zinc, preferably a zinc-cysteine complex, for a period of least at least 30 days.

15. A gastro-retentive pill that provides for sustained release of zinc, preferably a zinc-cysteine complex, in the upper gastrointestinal system.

16. A gastrointestinal implantable device that provides for sustained release of zinc, preferably a zinc-cysteine complex, in the upper gastrointestinal system.

17. A formulation, optionally a powder, for consumption with food that comprises zinc, preferably a zinc-cysteine complex, wherein the zinc competitively inhibits absorption of copper from the food.

18. A formulation, optionally a powder, for dissolution in a drinkable liquid, wherein the formulation contains at least 50 mg of zinc, preferably a zinc-cysteine complex per dose.

19. A pill, tablet or capsule for dissolution in a drinkable liquid, wherein the pill contains at least 50 mg of zinc, preferably a zinc-cysteine complex, per dose.

20. A deionized, drinkable liquid comprising at least 25 mg of zinc, preferably a zinc-cysteine complex, per dose.

21. A pill, tablet or capsule comprising a cholesterol-lowering agent and zinc, preferably a zinc-cysteine complex.

22. An orally-available, high dose, sustained-release formulation of zinc, preferably a zinc-cysteine complex.

23. The formulation of claim 22, wherein the dose is greater than 100 mg.

24. An orally-available pharmaceutical composition that comprises a non-ionic form of copper.

25. The composition of claim 24, wherein the copper is complexed with a digestible protein.

26. A two part pill, tablet or capsule, wherein one part contains zinc and is intended to release in the gastrointestinal tract or an animal or human, and the other part contains a copper chelator and is intended to chelate copper only after the zinc has induced metallotheionein production in the gastrointestinal tract.

27. A method of treating a disease characterized by an excessive level of homocysteine, comprising administering zinc, preferably a zinc-cysteine complex, or optionally a formulation of folic acid and zinc, preferably a zinc-cysteine complex, or folic acid and a copper chelator or a copper complex.

28. A pharmaceutical composition comprising zinc oxide weighing 1.0 mg per dose.

29. A method of treating a copper-mediated disease in an animal or human by administration of tetrathiomolybdate packaged under conditions of low moisture and oxygen levels.

30. A tablet, pill or capsule comprising zinc, preferably a zinc-cysteine complex, that dissolves or disintegrates at different rates in different locations of the gastrointestinal tract of an animal or human, so as to optimize induction of matrix metallotheionein in the gastrointestinal tract to efficiently block the potential absorption of copper that may be contained in such water.

31. A method of treatment of a copper-mediated disease in an animal or human by administration of an induction dose of a copper chelator and folic acid and/or zinc, or a zinc-acetate complex.

32. The method of claim 31, wherein the chelator is ammonium tetrathiomolybdate.

33. A pharmaceutical composition comprising zinc, preferably a zinc-cysteine complex, and folic acid.

34. A pellet comprising zinc, wherein the zinc is selected from the group of zinc, a zinc-cysteine complex, zinc acetate or another zinc salt, and wherein the density of the pellet is greater than 2 g/cm³, and wherein the pellet is capable of being retained in the rugae of the stomach of an animal or human.

35. The pellet of claim 34, wherein the pellet contains an enteric coating to delay release of its contents until after it has transgressed the stomach.

36. The pellet of claim 35, wherein the pellet is also coated with one or more layers of bioadhesive and/or mucoadhesive polymers.

37. A pharmaceutical composition comprising zinc, preferably a zinc-cysteine complex, and an iron supplement.

38. A method of treating schizophrenia comprising administration of a copper complexor, chelator, or blocker, and folic acid and/or zinc or a zinc-cysteine complex.

39. A prodrug composition of zinc, a zinc-cysteine complex, tetrathiomolybdate, trientine, d-penicillamine or other copper chelator, copper complexor, or blocker of copper absorption, wherein the prodrug preferably is cleaved, released or activated in vivo in a manner that optionally is dependent on presence and availability of non-ceruloplasmin bound copper or metalloprotein associated with an elevated level of non-ceruloplasmin bound systemic copper in the gastrointestinal tract or serum of a patient.

40. A prodrug of zinc, a zinc-cysteine complex, tetrathiomolybdate, trientine, d-penicillamine or other copper chelator, copper complexor, or blocker of copper absorption, wherein the prodrug preferably is released preferentially in the liver based upon contact with enzymes or proteins preferentially expressed in the liver.

41. A selective copper chelator in a pharmaceutical formulation suitable for oral administration to a mammal, wherein the chelator is capable of chelating copper in the gastrointestinal tract of the mammal.

42. The chelator of claim 41, wherein the chelator has a systemic bioavailability of less than 2%.

43. A pill, tablet or capsule suitable for oral administration to an animal or human comprising one or more microspheres, wherein the microspheres comprise one or more selective copper chelators capable of chelating copper in the gastrointestinal tract of the mammal.

44. A method of treating copper toxicosis in an elderly animal or human attributable to impaired hepatic function by the administration of a copper chelator.

45. The method of claim 44, further comprising the administration of an anti-inflammatory agent or anti-fibrotic agent to improve excretion of copper from the liver of an elderly animal or human.

46. The method of claim 45, wherein the anti-inflammatory agent is ursodiol.

47. A nutritional supplement that is low in copper and high in zinc, preferably a zinc-cysteine complex.

48. A method of selecting a dosage level of a copper chelator that is based upon a direct measurement of non-ceruloplasmin bound copper in a an animal or human.

49. A method of selecting a dosage level of a copper chelator that is based upon a measurement of copper chaperone for Cu/Zn superoxide dismutase in a an animal or human.

50. A formulation of enteric coated sustained release zinc in combination with ascorbic acid.

51. The formulation of claim 50, further comprising folic acid.

52. A method of selecting a dosage of a copper chelator that comprises determining the available free copper in the body of a patient and selecting a maintenance dosage that achieves a targeted range of free copper based upon extrapolation.

53. A method of selecting a dosage of a copper chelator that comprises determining the available free copper in the body of a patient by a direct measurement of free copper in the serum and further comprises selection of a maintenance dosage that will achieve a targeted range of free copper in the body based upon extrapolation.

54. A method of selecting a dosage of copper chelator that comprises determining the serum level of copper chaperone for Cu/Zn superoxide dismutase by a direct measurement of such protein in the serum, saliva or urine of an animal or human and further comprises the selection of a maintenance dosage that will achieve a targeted range of free copper based upon extrapolation.

55. A method of selecting a dosage of a copper chelator that comprises determining the level of isoprostanes in the serum, urine or saliva of an animal or human and selecting a maintenance dosage that will achieve a targeted range of free copper based upon extrapolation.

56. A method of treating conditions of hyperhomocysteine comprising administration of zinc.

57. A method of treating schizophrenia, Huntington's Disease or amyotrophic lateral silerosis comprising administration of zinc, preferably a zinc-cysteine complex.

58. A pharmaceutical composition comprising a copper supplement, wherein the supplement is complexed with a substance found in the breast milk of humans or animals.

59. The composition of claim 58, wherein the substance is whey.

60. The composition of claim 58, wherein the substance is a lipid.

61. The composition of claim 58, wherein the substance is a protein.

62. The composition of claim 58, wherein the substance is casein.

63. The composition of claim 58, further comprising zinc, preferably a zinc-cysteine complex.

64. The composition of claim 58, further comprising a zinc salt.

65. The composition of claim 58, further comprising gastroretentive zinc particles.

66. A composition comprising Cu⁶³ complexed with proteins.

67. The composition of claim 66, further comprising zinc, preferably a zinc-cysteine complex.

68. A composition comprising Cu⁶⁵ complexed with proteins.

69. The composition of claim 68, further comprising zinc, preferably a zinc-cysteine complex.

70. A pill, tablet or capsule comprising zinc, preferably a zinc-cysteine complex, and a hepatic function agent, preferably a bile acid binding agent and/or a copper reducing agent.

71. The pill, tablet or capsule of claim 70, wherein the hepatic function agent is ursodiolic acid.

72. A tablet comprising zinc, preferably a zinc-cysteine complex, and a hepatic function agent, preferably a bile acid binding agent and/or a copper reducing agent.

73. The tablet of claim 72, wherein the hepatic function agent is ursodiolic acid.

74. A pharmaceutical composition comprising the stable copper isotopes Cu⁶⁵ or Cu⁶⁷ in a ratio of other than approximately 31 % and 69%, and a hepatic function agent.

75. The composition of claim 74, wherein the hepatic function agent is ursodiolic acid.

76. A method of diagnosing a metabolic copper disease comprising administering Cu⁶⁵ and Cu⁶⁷ to a human or animal and measuring the proportion of Cu⁶⁵ and Cu⁶⁷ bound to at least one protein.

77. The method of claim 76, wherein the protein is selected from the group consisting of ceruloplasmin, trancuprein, superoxide dismutase, albumin and peptides.

78. The method of claim 76, wherein the protein is a high kinetic binding protein.

79. The method of claim 76, wherein the protein in a low kinetic binding protein.

80. A method of adjusting treatment of a metabolic copper disease comprising administering Cu⁶⁵ and Cu⁶⁷ to a human or animal and measuring the proportion of Cu⁶⁵ and Cu⁶⁷ bound to at least one protein.

81. A method of treating Alzheimer's disease in a subject comprising administration of a composition which protects the subject from exposure to soluble forms of copper contained in drinking fluids.

82. The method of claim 81, wherein the composition comprises zinc, preferably a zinc-cysteine complex.

83. A method of treating Alzheimer's disease in a human or animal, comprising administration of high molecular weight, naturally-occurring proteins and biologic complexes.

84. A pharmaceutically acceptable formulation suitable for human or animal use that contains less than 120 mg of tetrathiomolybdate, optionally less than 100, less than 80, less than 40, less than 20, less than 10 or less than 5.

85. A method of treating disease in a subject which comprises administration of a dose of a thiomolybdate of between 2 μg to 20 mg per day.

86. A method of treating a CNS disease in a human or animal which comprises administration of agent capable of stabilizing free copper levels in the serum.

87. Pegalated forms of superoxide dismutase or liposomo, depo injection or other sustained release forms of copper complexing agents selected from the group consisting of superoxide dismutase, ceroplasmin, metallotheionein, transferrin, amyloid beta, apoe4 and CCS.

88. The composition of claim 84, wherein the tetrathiomolybdate is in a sustained release formulation.

89. A supplement that contains two or more trace metals in a sustained release formulation selected from the group consisting of zinc, copper, iron, calcium, molybdenum, and magnesium.

90. The composition of claim 86, wherein the trace metals are bound to natural or synthetic digestible carriers that further limit their release and digestion.

91. The composition of claim 87, wherein carriers are selected from the group consisting of whey, plant fiber, dried milk, infant formula, metallotheionein, and transferrin.

92. The pharmaceutical composition of claim 4, wherein the zinc is released in a time of between 1 to 24 hours, 2 to 24 hours, 3 to 24 hours, 4 to 24 hours, 5 to 24 hours, 6 to 24 hours, 8 to 24 hours or 12 to 24 hours.

93. A formulation of tetrathiothiomolybdate comprised predomininatly of large crystals to allow protection from the environment of gastric juice of the stomach.

94. A formulation of tetrathiothiomolybdate comprised of a heterogenous collection of crystal size to allow the smaller populations of crystals the opportunity to complex with copper in foodstuffs in the meal and the larger crystals protection from the environment of gastric juice of the stomach to allow dissolution in the small intestine.

95. A formulation of tetrathiothiomolybdate comprised predominnaty of small crystals size to allow greater opportunity to complex with copper in foodstuffs in the meal.

96. A dose of tetrathiomolybdate comprised of predominantly large tetrathiomolybdate crystals given with food or away from food.

97. A dose of tetrathiomolybdate comprised of a heterogeneous size range of tetrathiomolybdate crystals given with food or away from food.

98. A dose of tetrathiomolybdate comprised of predominantly small tetrathiomolybdate crystals given with food or away from food.

99. A method of any of claims 96-98, where the dose of tetrathiomolybdate is 0.01-4 mg/kg/day in a mammal.

100. A formulation method to protect tetrathiomolybdate from exposure to the environment of the stomach.

101. A formulation whereby tetrathiomolybdate is encapsulated by enteric coating.

102. A formulation whereby tetrathiomolybdate is prepared by lyophilization of a solution and the resulting powder is compressed in to a tablet and enteric coated.

103. A formulation of zinc containing copper reducing agent and an enteric coated tetrathiomolybdate powder.

104. A formulation of tetrathiomolybdate and mineral oil in an encapsulated enteric coated delivery system for release in the small intestine.

105. A method of using a anti-copper or copper malabsorption agent to treat a disease in a human or animal selected from the group of Alzheimer's disease, Parkinson's disease, amyotrophic lateral sclerosis (ALS), mild cognitive impairment, dementia, Huntington's disease, Pick's disease Behcet's disease, schizophrenia, bipolar disorder, psychosis depression, autism, multiple sclerosis.

106. The method of claim 105, wherein the anti-copper or copper malabsorption agent is selected from the group consisting of zinc, a zinc-amino acid complex, a zinc salt, a non-salt zinc amino acid complex, a zinc cysteine complex, a non-salt zinc cysteine complex, zinc monocysteine, a thiomolybdate, tetrathiomolybdate, ammonium tetrathiomolybdate, trientine, penicillamine, clioquinol, phytic acid, citric acid, deferasirox or any combination thereof.

107. The method of claim 105, where in the anti-copper or copper malabsorption agent is used in combination with a second agent selected from the group of an acetylcholine esterase inhibitor, NMDA receptor antagonist, antidepressant, antipsychotic and cholesterol lowering agent or any combination thereof.

108. A method of using a anti-copper or copper malabsorption agent to treat a disease in a human or animal selected from the group of a disease associated with abnormal accumulation of copper associated proteins in the body, juvenile and sporadic inclusion body myositis myositis of the elderly, cardiovascular disease, atherosclerosis, stroke, and peripheral.

109. A pharmaceutical composition suitable for oral administration in a human or animal that containing thiomolybdate crystals of a size substantially between 100 and 1,000 microns in diameter at their widest point.

110. The composition of claim 109, wherein thiomolybdate crystals have an average size of approximately 350 microns.

111. A pharmaceutical composition suitable for oral administration in a human or animal that containing thiomolybdate crystals of an individual weight of approximately 0.3 picograms.

112. A method of controlling the dissolution, release or absorption of an oral dosage form of thiomolybdate in the gastrointestinal tract of a human or animal by varying the size or distribution of the thiomolybdate crystals contained therein.

113. A sustained release formulation of a thiomolybdate comprising a capsule tablet or other delivery form suitable for oral delivery with or away from food whereby thiomolybdate crystals larger than 50 microns in size are immediately released in the stomach.

114. The composition of claim 113, wherein the thiomolybdate crystals are of sufficient size so as to permit at least 25% or more of the thiomolybdate material is capable of transiting the gastrointestinal conditions of the stomach as a thiomolybdate and bind endogeneous copper secreted into the jejunum and intestines via the bile.

115. A pharmaceutical composition comprising an immediate release capsule containing thiomolybdate crystals, particles microparticles enterically coated microparticles capable of binding copper contained in food as well as copper endogenously secreted into the jejunum and intestines via the bile.

116. A method of treating Wilson's disease in a patient by administration of an oral dosage form described in anyone of claims 109, 110, 111, 112, 113, 114 or 115.

117. The method of claim 116, wherein Wilson's disease is neurologically presenting Wilson's disease.

118. The method of claim 117, wherein a thiomolybdate is administered to patient in need thereof orally three times per day with meals and three times a day away from meals.

119. The method of claim 118, wherein the thiomolybdate is administered to patient in need thereof orally three times per day with meals and once a day at bedtime away from food at a dose equivalent to the total daily dose administered with meals.

120. The method of claim 118, wherein the thiomolybdate administered orally with meals is swallowed immediately prior to the first bite of food.

121. The method of claim 119, wherein the thiomolybdate administered orally with meals is swallowed immediately prior to the first bite of food.

122. The method of claim 118, wherein the thiomolybdate administered orally with meals is swallowed substantially with the first bite of food.

123. The method of claim 119, wherein the thiomolybdate administered orally with meals is swallowed substantially with the first bite of food.

124. The method of claim 118, wherein the thiomolybdate administered orally with meals is swallowed substantially within the first fifteen minutes of the first bite of the meal.

125. The method of claim 119, wherein the thiomolybdate administered orally with meals is swallowed substantially within the first fifteen minutes of the first bite of the meal.

126. The method of claim 118, wherein the thiomolybdate administered orally with meals is swallowed immediately after such meal.

127. The method of claim 119, wherein the thiomolybdate administered orally with meals is swallowed immediately after such meal.

128. The method of claim 118, wherein the thiomolybdate administered orally with meals is consumed in a form either sprinkled on or mixed with food or drink consumed during such meal.

129. The method of claim 119, wherein the thiomolybdate administered orally with meals is consumed in a form either sprinkled on or mixed with food or drink consumed during such meal.

130. The method of claim 118, wherein the individual dosages of the thiomolybdate are approximately 18 to 22 mg per dose.

131. The method of claim 119, wherein the individual dosages of the thiomolybdate are approximately 18 to 22 mg per dose.

132. The method of claim 120, wherein the treatment is continued for approximately 8 weeks.

133. The method of claim 121, wherein the treatment is continued for approximately 8 weeks.

134. The method of claim 119, wherein the treatment is continued for approximately 16 weeks and the daily dose is 120 per day for the first two weeks and 60 mg per day for an additional 14 weeks.

135. The method of claim 120, wherein the treatment is continued for approximately 16 weeks and the daily dose is 120 per day for the first two weeks and 60 mg per day for an additional 14 weeks.

136. A method of reducing the incidence of leukapenia or anemia in a neurologically presenting Wilson's disease patient receiving oral thiomolybdate therapy wherein the serum ceruloplasmin levels of said patient are monitored at least once per month and dosage is abated or reduced if the serum ceruloplasmin levels of said patient are less than 10 mg/dl.

137. A composition comprising a bottle or other container substantially purged of oxygen via inert gas containing at least one desiccant and capsules, tablets or other oral dosage forms containing thiomolybdate suitable for oral administration.

138. A method of improving the shelf life and stability of the composition of claim 137 by refrigerating such composition at approximately 4° C.

139. The method claim 138, wherein the composition is thawed to room temperature prior to opening by a patient so as to reduce condensation and improve stability.

140. The method of claim 138, wherein after opening the composition is maintained at room temperature so as to reduce condensation and improve stability.

141. The composition of claim 38, wherein such composition contains a unit count of 6, 10, 12, 18, 24, 30, 36, 42, 48, 50, 96 or 100 capsules, tablets or other oral dosage forms.

142. An enteric coated capsule tablet or other oral dosage form containing an immediate release zinc, zinc salt, zinc amino acid complex, zinc cysteine complex, nonsalt zinc cyteine complex or zinc monocytsteine designed to release its contents after transiting the stomach.

143. A multivitamin in which redox active minerals including as copper or iron are complexed with digestible protein, fiber, or other natural or synthetic material so as to minimize the potential bolus flux of such metals into the serum of patients.

144. A multivitamin in which redox active minerals including as copper or iron are in a sustained release form so as to minimize the potential bolus flux of such metals in a free into the serum of patients.

145. A capsule containing a thiomolybdate suitable for oral administration in a human and immediate release in the stomach wherein the capsule has a water content of approximately 6% or less.

146. The composition of claim 145, wherein the capsule is comprised of a material selected from the group of methyl cellulose carrageenan, polyesters, poly(nonhalogenated hydrocarbons), poly(halogenated hydrocarbons), poly(halogenated polyethers), polymers formed from dienes, poly(higher alkylene oxides), polyamides, polysiloxanes, polysilanes, poly(acrylonitriles), poly(lower alkylene oxides), acrylate polymers, polyacrylic acids and alternating copolymers, poly(ethylene terephthalate), poly(butylene terephthalate), poly(trimethylene terephthalate), poly(ethylene naphthalate), polyethylene, polypropylene, polystyrene, polyisobutylene, polymethylpentene, poly(tetrafluoro ethylene), poly(chlorotrifluoro ethylene), poly(vinyl chloride), poly(vinyl fluoride), poly(vinyl bromide), poly(vinyl iodide), poly(vinylidene chloride), poly(vinylidene fluoride), poly(vinylidene bromide), poly(vinylidene iodide), chlorinated polyether, polybutadiene, poly(dicyclopentadiene), poly(butylene oxide), poly(propylene oxide), poly(2,6-dimethylphenylene oxide), nylons, poly(dimethyl siloxane), poly(methylphenyl siloxane), poly(diphenyl siloxane), poly(methylphenyl silane), poly(acrylonitrile), poly(ethylene oxide), poly(methyl acrylate), poly(ethyl acrylate), polymethacrylic acid, polyethylacrylic acid, poly(methyl methacrylate), poly(ethyl methacrylate), and alternating copolymers, block copolymers, random copolymers, graft copolymers, terpolymers, block terpolymers, and random terpolymers thereof, and/or polyethylene, polypropylene, polystyrene, and combinations of any of the foregoing.

147. A capsule containing a thiomolybdate suitable for oral administration in a human and immediate release in the stomach wherein the capsule has approximately no water content.

148. The composition described in claim 146 that are enteric coated with a moisture impermeable material.

149. The composition described in claim 147 that are enteric coated with a moisture impermeable material.

150. The composition described in claim 146, including an excipient having a water content of 1% or less.

151. The composition described in claim 147, including an excipient having a water content of 1% or less.

152. The composition described in claim 148, including an excipient having a water content of 1% or less.

153. The composition of claim 150, wherein the excipient is anhydrous lactose.

154. The composition of claim 151, wherein the excipient is anhydrous lactose.

155. The composition of claim 152, wherein the excipient is lactose monohydrate.

156. The pellet of claim 35, wherein the enteric coating is pH dependent and selected to prevent degradation of the coating and release of the zinc while the pellet is still in the low pH conditions of the stomach and/or pyloric region (pH of 1.2-3.5).

157. The pellet of claim 35, wherein the enteric coating is selected from the group comprising cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropyhnethyl cellulose succinate and carboxymethylcellulose sodium; acrylic acid polymers and copolymers, preferably formed from acrylic acid, methacrylic acid, methyl acrylate, ammonio methylacrylate, ethyl acrylate, methyl methacrylate and/or ethyl methacrylate (“Eudragit”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac).

158. The formulation of claim 17 or 18, wherein the formulation further comprises an acetyl cholinesterase inhibitor.

159. The formulation of claim 17 or 18, wherein the formulation further comprises an NMDA antagonist.

160. The formulation of claim 159, wherein the NMDA antagonist is memantine or flupirtine.

161. The formulation of claim 17 or 18, wherein the formulation further comprises an agent capable of improving hepatic and biliary clearance functions.

162. The formulation of claim 161, wherein the agent is a statin.

163. The formulation of claim 161, wherein the agent is an RXR specific ligand.

164. The formulation of claim 161, wherein the agent is tetrathiomolybdate.

165. The method of claim 29, wherein the disease is a neurological disease.

166. The method of claim 31, wherein the disease is a neurological disease.

167. The method of claim 29, wherein the neurological disease is selected from the group consisting of Wilson's Disease, Alzheimer's Disease, Parkinson's Disease, Amyotrophic Lateral Sclerosis, Menkes Disease, progressive supranuclear palsy, dementia, mild cognitive impairment, Huntington's Disease, autism, postencephaltic Parkinson's Disease, Pick's Disease and schizophrenia.

168. The method of claim 31, wherein the neurological disease is selected from the group consisting of Wilson's Disease, Alzheimer's Disease, Parkinson's Disease, Amyotrophic Lateral Sclerosis, Menkes Disease, progressive supranuclear palsy, dementia, mild cognitive impairment, Huntington's Disease, autism, postencephaltic Parkinson's Disease, Pick's Disease and schizophrenia.

169. The method of claim 29, wherein the disease is a neuromuscular disease.

170. The method of claim 31, wherein the disease is a neuromuscular disease.

171. The method of claim 31, wherein the neuromuscular disease is selected from the group consisting of juvenile and sporadic inclusion body myositis and myositis of the elderly.

172. The method of claim 29, wherein the neuromuscular disease is selected from the group consisting of juvenile and sporadic inclusion body myositis and myositis of the elderly.

173. The method of claim 31, wherein the disease is an autoimmune disease.

174. The method of claim 29, wherein the disease is an autoimmune disease.

175. The method of claim 31, wherein the disease is multiple sclerosis.

176. The method of claim 29, wherein the disease is multiple sclerosis.

177. The method of claim 31, wherein the disease is a cardiovascular disease.

178. The method of claim 29, wherein the disease is a cardiovascular disease.

179. The method of claim 31, wherein the cardiovascular disease is selected from the group consisting of atherosclerosis, stroke and peripheral vascular disease.

180. The method of claim 29, wherein the cardiovascular disease is selected from the group consisting of atherosclerosis, stroke and peripheral vascular disease.

181. The method of claim 31, wherein the disease is an inflammatory disease.

182. The method of claim 29, wherein the disease is an inflammatory disease.

183. The method of claim 31, wherein the disease is a fibrotic disease.

184. The method of claim 29, wherein the disease is a fibrotic disease.

185. The method of claim 31, wherein the disease is a liver disease.

186. The method of claim 29, wherein the disease is a liver disease.

187. The method claim 31, wherein the liver disease is selected from the group consisting of nonalcoholic steatohepatitis and non-viral hepatitis.

188. The method claim 29, wherein the liver disease is selected from the group consisting of nonalcoholic steatohepatitis and non-viral hepatitis.

189. The method of claim 31, wherein the disease is diabetes.

190. The method of claim 29, wherein the disease is diabetes.

191. The method of claim 31, wherein the disease is geriatric-related impaired copper excretion.

192. The method of claim 29, wherein the disease is geriatric-related impaired copper excretion.

193. A method of treating intraday fluctuations in levels of free copper in the serum and CNS, which comprises (a) administration of tetrathiomolybdate packaged under conditions of low moisture and oxygen levels, or (b) administration of an induction dose of a copper chelator and folic acid and/or zinc, or a zinc-acetate complex; wherein the copper chelator preferably is ammonium tetrathiomolybdate.

194. A method of maintaining a healthy copper status in elderly patients comprising normalizing hepatic copper excretion by the administration of a copper chelator, optionally further comprising the administration of an anti-inflammatory or anti-fibrotic agent to improve excretion of copper from the liver of an elderly patient, wherein the anti-inflammatory agent preferably is ursodiol.

195. The method of claim 85, wherein the thiomolybdate is selected from the group consisting of tetrathiomolybdate, trithiomolybdate, dithiomolybdate and monothiomolybdate.

196. The method of claim 86, wherein the agent is tetrathiomolybate.

197. The method of claim 86, wherein the agent is sustained release tetrathiomolybdate.

198. The method of claim 86, wherein the agent is a depo injection formulation of a copper binding agent bound to a peptide.

199. The method of claim 86, wherein the agent is a pegalated peptide containing a cysteine residue.

200. The method of claim 199, wherein the pegalated peptide is pegalated superoxide dismutase.

201. The supplement of claim 89, wherein the supplement further comprises a copper malabsorption agent.

202. The supplemental of claim 201, wherein the copper malabsorption agent is zinc.

203. The supplemental of claim 89, wherein the supplement is incorporated within a sustained release microparticle or matrix.

204. A capsule which comprises a thiomolybdate.

205. The capsule of claim 204, wherein the thiomolybdate is selected from the group consisting of tetrathiomolybdate, trithiomolybdate, dithiomolybdate and monothiomolybdate.

206. The capsule of claim 204, wherein the thiomolybdate is a complex thiomolybdate.

207. The capsule of claim 204, wherein the capsule has a very low water content.

208. The capsule of claim 204, wherein the capsule contains an excipient that has a low water content.

209. The capsule of claim 204, wherein the capsule is packaged in modified atmosphere packaging.

210. The capsule of claim 209, wherein the packaging is an impervious foil pouch.

211. The capsule of claim 209, wherein the packaging is a blister pack.

212. The capsule of claim 209, wherein the packaging is purged and sealed with an inert gas.

213. The capsule of claim 212, wherein the gas is nitrogen or argon.
